(19) United States
(12) Patent Application Publication
(10) Pub. No.: US 2022/0396832 A1
(43) Pub. Date: Dec. 15, 2022

(54) COMPOSITIONS AND METHODS FOR SEQUENCING BY SYNTHESIS

(71) Applicants: Illumina Cambridge Limited, Cambridge (GB); Illumina, Inc., San Diego, CA (US)

(72) Inventors: Angelica Mariani, Cambridge (GB); Antoine Francais, Cambridge (GB); Frederick James Topping, Cambridge (GB); Christopher Winnard, Cambridge (GB); Philip Balding, Cambridge (GB); Chol Steven Yun, San Diego, CA (US); Patrizia Iavicoli, San Diego, CA (US)

(21) Appl. No.: 17/748,498

(22) Filed: May 19, 2022

Related U.S. Application Data
(60) Provisional application No. 63/190,983, filed on May 20, 2021.

(51) Int. Cl. C12Q 1/6869

(52) U.S. Cl. CPC C12Q 1/6869

(57) ABSTRACT

The present application relates to compositions and methods for sequencing by synthesis, where one or more palladium scavengers were used to improve sequencing metrics such phasing and prephasing values.

### INCORPORATION BY REFERENCE TO PRIORITY APPLICATION

The present application claims the benefit of priority to U.S. Provisional Application No. 63/190,983, filed May 20, 2021, which is incorporated by reference in its entirety.

### BACKGROUND

### Field

The present disclosure generally relates to polynucleotide sequencing methods, compositions, and kits for sequencing.

### Description of the Related Art

Advances in the study of molecules have been led, in part, by improvement in technologies used to characterize the molecules or their biological reactions. In particular, the study of the nucleic acids DNA and RNA has benefited from developing technologies used for sequence analysis and the study of hybridization events.

An example of the technologies that have improved the study of nucleic acids is the development of fabricated arrays of immobilized nucleic acids. These arrays consist typically of a high-density matrix of polynucleotides immobilized onto a solid support material. See, e.g., Fodor et al., Trends Biotech. 12: 19-26, 1994, which describes ways of assembling the nucleic acids using a chemically sensitized glass surface protected by a mask, but exposed at defined areas to allow attachment of suitably modified nucleotide phosphoramidites. Fabricated arrays can also be manufactured by the technique of “spotting” known polynucleotides onto a solid support at predetermined positions (e.g., Stimpson et al., Proc. Natl. Acad. Sci. 92: 6379-6383, 1995).

One way of determining the nucleotide sequence of a nucleic acid bound to an array is called “sequencing by synthesis” or “SBS”. This technique for determining the sequence of DNA ideally requires the controlled (i.e., one at a time) incorporation of the correct complementary nucleotide opposite the nucleic acid being sequenced. This allows for accurate sequencing by adding nucleotides in multiple cycles as each nucleotide residue is sequenced one at a time, thus preventing an uncontrolled series of incorporations from occurring. The incorporated nucleotide is read using an appropriate label attached thereto before removal of the label moiety and the subsequent next round of sequencing.

In order to ensure that only a single incorporation occurs, a structural modification (“protecting group” or “blocking group”) is included in each labeled nucleotide that is added to the growing chain to ensure that only one nucleotide is incorporated. After the nucleotide with the protecting group has been added, the protecting group is then removed, under reaction conditions which do not interfere with the integrity of the DNA being sequenced. The sequencing cycle can then continue with the incorporation of the next protected, labeled nucleotide. To be useful in DNA sequencing, nucleotides, which are usually nucleotide triphosphates, generally require a 3′ hydroxy blocking group so as to prevent the polymerase used to incorporate it into a polynucleotide chain from continuing to replicate once the base on the nucleotide is added.

Various compositions are employed at each step of a cycle of sequencing. For example, an incorporation composition comprising a polymerase and one or more different types of nucleotides are employed during the incorporation step. A scan composition that may include, among other things, an antioxidant to protect the polynucleotides from photo-induced damage during the detection step when, for example, the nucleotides include fluorophore labels for detection. A deblocking composition that includes reagents for cleaving the blocking moiety (e.g., the 3′ hydroxy blocking group) from the nucleotide incorporated is employed during the deblocking step. Cleavage reagents such as palladium (Pd) catalysts prepared from palladium complexes in the presence of water-soluble phosphine ligand(s) has been reported in the deblocking composition, for example, U.S. Publication No. 2020/0216891 and U.S. Ser. No. 63/042,240, each of which is incorporated by reference in its entirety. Pd has the capacity to stick on DNA, mostly in its inactive Pd(II) form, which may interfere with the binding between DNA and polymerase, causing increased phasing. A post-cleavage wash composition that includes a Pd scavenger compound may be used following the deblocking step. For example, PCT Publication No. WO 2020/126593 discloses Pd scavengers such as 3,3′-dithiodipropionic acid (DDPA) and lipoic acid (LA) may be included in the scan composition and/or the post-cleavage wash composition. The use of these scavengers in the post-cleave washing solution has the purpose of scavenging Pd(0), converting Pd(0) to the inactive Pd(II) form, thereby improving the prephasing value and sequencing metrics, reducing signal degrade, and extend sequencing read length. However, there exists a continued demand for developing compositions for use at each step of the sequencing to optimize performance.

### SUMMARY

Some aspect of the present disclosure relates to a method for determining the sequences of a plurality of target polynucleotides, comprising:

(a) contacting a solid support with sequencing primers under hybridization conditions, wherein the solid support comprises a plurality of different target polynucleotides immobilized thereon; and the sequencing primers are complementary to at least a portion of the target polynucleotides;

(b) contacting the solid support with a first aqueous solution comprising DNA polymerase and one or more of four different types of nucleotides (e.g., dATP, dCTP, dGTP, and dTTP or dUTP) under conditions suitable for DNA polymerase-mediated primer extension, wherein each of the nucleotides comprises a 3′ blocking group having the structure

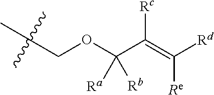

attached to the 3′ oxygen of the nucleotide;

(c) incorporating one type of nucleotides into the sequencing primers to produce extended copy polynucleotides;

(d) performing one or more fluorescent measurements of the extended copy polynucleotides; and

(e) removing the 3′ blocking group of the incorporated nucleotides with a palladium catalyst;

wherein at least a portion of remaining palladium catalyst is inactivated by one or more palladium scavengers, wherein at least one palladium scavenger comprises one or more allyl moieties selected from the group consisting of —O-allyl, —S-allyl, —NR-allyl, and —N⁺RR′-allyl, and combinations thereof; and

wherein each of R^{a}, R^{b}, R^{c}, R^{d} and R^{e} is independently H, halogen, unsubstituted or substituted C₁-C₆ alkyl, or C₁-C₆ haloalkyl; R is H, unsubstituted or substituted C₁-C₆ alkyl, unsubstituted or substituted C₂-C₆ alkenyl, unsubstituted or substituted C₂-C₆ alkynyl, unsubstituted or substituted C₆-C₁₀ aryl, unsubstituted or substituted 5 to 10 membered heteroaryl, unsubstituted or substituted C₃-C₁₀ carbocyclyl, or unsubstituted or substituted 5 to 10 membered heterocyclyl; and R′ is H, unsubstituted C₁-C₆ alkyl or substituted C₁-C₆ alkyl. In some embodiments, the remaining palladium catalyst (in the form of Pd(0) and/or Pd(II)) is inactivated by one or more palladium scavengers. In some embodiments, each of the nucleotide in the first aqueous solution comprises a 3′ blocking group having the structure

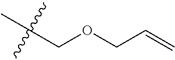 [SMILES: *COCC=C]

attached to the 3′ oxygen of the nucleotide. In some embodiments, one or more of the nucleotides in the first aqueous solution comprises a fluorescent label. In some embodiments, steps (b) to (e) are repeated until a sequence of a portion of the target polynucleotide is determined.

Some aspect of the present disclosure relates to a kit for use with a sequencing apparatus, comprising: one or more different types of nucleotides, wherein each of the nucleotides comprises a 3′ hydroxy blocking group having the structure

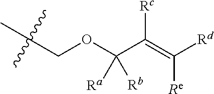

attached to the 3′ oxygen of the nucleotide, wherein each of R^{a}, R^{b}, R^{c}, R^{d} and R^{e} is independently H, halogen, unsubstituted or substituted C₁-C₆ alkyl, or C₁-C₆ haloalkyl; and one or more palladium scavengers, wherein at least one palladium scavenger comprises one or more allyl moieties selected from the group consisting of —O-allyl, —S-allyl, —NR-allyl, and —N⁺RR′-allyl as described herein, and combinations thereof. In some embodiments, each of the nucleotide in the kit comprises a 3′ hydroxy blocking group having the structure

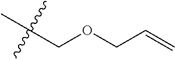 [SMILES: *COCC=C]

attached to the 3′ oxygen of the nucleotide.

Some other aspects of the present disclosure relate to a cartridge for use with a sequencing apparatus, comprising a plurality of chambers, each chamber contains a single composition, wherein the kit described herein is for use in one of the chambers, for example for use in an incorporation step of the sequencing method described herein. Additional compositions may include but not limited to: a scan buffer composition comprising one or more antioxidant and optionally a scavenger; a cleavage composition comprising Pd reagents for removing the 3′ hydroxy blocking group of the incorporated nucleotide and/or the fluorescent label; and a wash buffer, which may contain one or more additional Pd scavengers to inactivate the remaining Pd catalyst (in the form of Pd(0) and/or Pd(II)) after the cleavage reaction, prior to the next cycle of sequencing.

### BRIEF DESCRIPTION OF THE DRAWINGS

FIG. 1 is a bar chart illustrating the prephasing value of a sequencing run when palladium scavenger Compound A is used in a post-cleavage wash solution in various concentrations as compared to a standard post-cleavage wash using lipoic acid.

FIG. 2A is a line chart illustrating the kinetic evaluation of various palladium scavengers in a kinetic assay as compared to no scavenger in the same kinetic assay.

FIG. 2B is a magnified line chart of the circled area of FIG. 2A comparing several palladium scavengers with lipoic acid.

FIG. 3 is a bar chart illustrating the percent prephasing values of a sequencing run on Illumina's iSeq™ platform when incorporation mixtures containing Pd(0) scavenger Compound B or C were compared to a standard incorporation mixture without a Pd(0) scavenger but utilizing a lipoic acid post cleavage wash step.

FIG. 4 illustrate the sequencing metrics (phasing value, prephasing value, error rate and Q30 respectively) of sequencing runs on Illumina's iSeq™ platform utilizing several incorporation mixtures containing a Pd(0) scavenger Compound B or C as compared to a standard incorporation mixture without any Pd scavenger with two different incorporation times (24 seconds and 19 seconds).

FIG. 5 illustrates the mean percent prephasing values for Read 1 and Read 2 of sequencing runs on Illumina's iSeq™ platform using Pd(0) scavenger Compound B, as compared to Pd(0) scavenger Compound O (DADMAC).

FIG. 6 illustrates the mean percent phasing values for Read 1 and Read 2 of sequencing runs on Illumina's iSeq™ platform using Pd(II) scavengers L-cysteine or sodium thiosulfate, as compared to those without any Pd(II) scavengers.

### DETAILED DESCRIPTION

Some aspects of the present disclosure relate to methods for improving sequencing metrics in nucleic acid sequencing, for example, the phasing and prephasing values in sequencing by synthesis. In particular, the sequencing method described herein involves the use of a palladium (Pd) catalyst to cleave the 3′ hydroxy blocking group of an incorporated nucleotide prior to the next incorporation cycle. Pd catalysts have the tendency to stick on nucleic acid such as DNA (e.g., the copy polynucleotide) during sequencing by synthesis, either in the inactivated Pd(II) form or the catalytically active Pd(0) form. When Pd(II) sticks on DNA, it may slow down the binding of the growing polynucleotide chain with the DNA polymerase, creating phasing. When excess Pd(0) sticks on the DNA, it may cleave the 3′ hydroxy blocking group of the nucleotide in the incorporation mix prior to the incorporation and/or fluorescent measurement(s) steps. When this happens, it creates prephasing. To improve the sequencing metrics, the sequencing method typically includes a post cleavage washing step to remove any remaining Pd catalyst. However, a simple wash buffer may not be able to completely suppress the activity of the residual Pd catalyst. In addition, one or more palladium scavengers may be included in one or more buffer solutions used after the incorporation step (e.g., either in the post cleavage washing buffer or in the scan buffer) to inactivate the residual palladium catalyst prior to the next cycle. Lipoic acid has been used as an effective palladium scavenger to inactivating the active Pd(0) catalyst by oxidizing it to Pd(II) form. However, due to its oxidative nature, lipoic acid is incompatible with other sequencing reagents. As a result, the use of lipoic acid requires a separate washing step to remove any excess lipoic acid before the next cycle of sequencing.

Certain aspects of the present disclosure relate to employing alternative palladium scavengers in several steps of sequencing by synthesis, where at least one palladium scavenger comprises one or more allyl moieties (e.g., —O-allyl, —S-allyl, —NR-allyl, or —N⁺RR′-allyl), or combinations thereof), acting as a competitive substrate to consume any residual Pd(0) sticking on the nucleic acid. The sequencing methods described herein substantially improve the sequencing metrics (e.g., reduce phasing and prephasing values) and may also reduce the sequencing time for each cycle by certain eliminating post-cleavage treatment step.

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where a range of values is provided, it is understood that the upper and lower limit, and each intervening value between the upper and lower limit of the range is encompassed within the embodiments.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffN Fully functionalized nucleotide
ffA Fully functionalized “A” nucleotide
ffC Fully functionalized “C” nucleotide
ffT Fully functionalized “T” nucleotide
ffG Fully functionalized “G” nucleotide
IMX Incorporation mix or Incorporation mixture
RT Room temperature
SBS Sequencing by Synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, “inactivate” or “inactivating” a palladium catalyst include but not limited to the following several mechanisms of using a palladium scavenger: (1) the palladium scavenger may act as a competitive substrate to consume any residual active Pd(0) sticking on the nucleic acid; (2) the palladium scavenger may act as an oxidizer to convert the active Pd(0) to the inactive Pd(II) form; and (3) the palladium scavenger may act as a competitive ligand to remove the Pd (e.g., Pd(0) or Pd(II)) sticking on the nucleic acid.

As used herein, any “R” group(s) represent substituents that can be attached to the indicated atom. An R group may be substituted or unsubstituted.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “aryloxy” refers to RO— in which R is an aryl, as defined above, such as but not limited to phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzoimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidinonyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(aryl)alkyl” refer to an aryl group, as defined above, connected, as a substituent, via an alkylene group, as described above. The alkylene and aryl group of an aralkyl may be substituted or unsubstituted. Examples include but are not limited to benzyl, 2-phenylalkyl, 3-phenylalkyl, and naphthylalkyl. In some embodiments, the alkylene is an unsubstituted straight chain containing 1, 2, 3, 4, 5, or 6 methylene unit(s).

As used herein, “(heteroaryl)alkyl” refer to a heteroaryl group, as defined above, connected, as a substituent, via an alkylene group, as defined above. The alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs. In some embodiments, the alkylene is an unsubstituted straight chain containing 1, 2, 3, 4, 5, or 6 methylene unit(s).

As used herein, “(heterocyclyl)alkyl” refer to a heterocyclic or a heterocyclyl group, as defined above, connected, as a substituent, via an alkylene group, as defined above. The alkylene and heterocyclyl groups of a (heterocyclyl)alkyl may be substituted or unsubstituted. Examples include but are not limited to (tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl. In some embodiments, the alkylene is an unsubstituted straight chain containing 1, 2, 3, 4, 5, or 6 methylene unit(s).

As used herein, “(carbocyclyl)alkyl” refer to a carbocyclyl group (as defined herein) connected, as a substituent, via an alkylene group. Examples include but are not limited to cyclopropylmethyl, cyclobutylmethyl, cyclopentylethyl, and cyclohexylpropyl. In some embodiments, the alkylene is an unsubstituted straight chain containing 1, 2, 3, 4, 5, or 6 methylene unit(s).

As used herein, “alkoxyalkyl” or “(alkoxy)alkyl” refers to an alkoxy group connected via an alkylene group, such as C₂-C₈ alkoxyalkyl, or (C₁-C₆ alkoxy) C₁-C₆ alkyl, for example, —(CH₂)₁₋₃-OCH₃.

As used herein, “—O-alkoxyalkyl” or “—O-(alkoxy)alkyl” refers to an alkoxy group connected via an —O-(alkylene) group, such as —O—(C₁-C₆ alkoxy) C₁-C₆ alkyl, for example, —O—(CH₂)₁₋₃-OCH₃.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

An “amino” group refers to a —NH₂ group. The term “mono-substituted amino group” as used herein refers to an amino (—NH₂) group where one of the hydrogen atom is replaced by a substituent. The term “di-substituted amino group” as used herein refers to an amino (—NH₂) group where each of the two hydrogen atoms is replaced by a substituent. The term “optionally substituted amino,” as used herein refer to a —NR_{A}R_{B} group where R_{A} and R_{B} are independently hydrogen, alkyl, cycloalkyl, aryl, heteroaryl, heterocyclyl, aralkyl, or heterocyclyl(alkyl), as defined herein.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “sulfo” group refers to a “—S(═O)₂OH” or “—SO₃H” group.

A “sulfonate” group refers to a “—SO₃⁻” group.

A “sulfate” group refers to “—SO₄⁻” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “O-carbamyl” group refers to a “—OC(═O)N(R_{A}R_{B})” group in which R_{A} and R_{B} can be the same as defined with respect to S-sulfonamido. An O-carbamyl may be substituted or unsubstituted.

An “N-carbamyl” group refers to an “ROC(═O)N(R_{A})—” group in which R and R_{A} can be the same as defined with respect to N-sulfonamido. An N-carbamyl may be substituted or unsubstituted.

An “O-thiocarbamyl” group refers to a “—OC(═S)—N(R_{A}R_{B})” group in which R_{A} and R_{B} can be the same as defined with respect to S-sulfonamido. An O-thiocarbamyl may be substituted or unsubstituted.

An “N-thiocarbamyl” group refers to an “ROC(═S)N(R_{A})—” group in which R and R_{A} can be the same as defined with respect to N-sulfonamido. An N-thiocarbamyl may be substituted or unsubstituted.

The term “propargylamine” as used herein, refers to an amino group that is substituted with a propargyl group (HC≡C—CH₂—). When propargylamine is used in the context as a bivalent moiety, it includes —C≡C—CH₂—NR_{A}— where R_{A} is hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

The term “propargylamide” as used herein, refers to a C-amido or N-amido group that is substituted with a propargyl group (HC≡C—CH₂—). When propargylamide is used in the context as a bivalent moiety, it includes —C≡C—CH₂—NR_{A}—C(═O)— or —C≡C—CH₂—C(═O)—NR_{A}, where R_{A} is hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

The term “allylamine” as used herein, refers to an amino group that is substituted with an allyl group (CH₂═CH—CH₂—). When allylamine is used in the context as a bivalent moiety, it includes —CH═CH—CH₂—NR_{A}—, where R_{A} is hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

The term “allylamide” as used herein, refers to a C-amido or N-amido group that is substituted with an allyl group (CH₂═CH═CH₂—). When allylamide is used in the context as a bivalent moiety, it includes —CH═CH═CH₂—NR_{A}—C(═O)— or —CH═CH—CH₂—C(═O)—NR_{A}—, where R_{A} is hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

The term “alkylamino” or “(alkyl)amino” refers to an amino group wherein one or both hydrogen is replaced by an alkyl group.

An “(alkoxy)alkyl” group refers to an alkoxy group connected via an alkylene group, such as a “(C₁-C₆ alkoxy) C₁-C₆ alkyl” and the like.

The term “hydroxy” as used herein refers to a —OH group.

The term “cyano” group as used herein refers to a “—CN” group.

The term “azido” as used herein refers to a —N₃ group.

When a group is described as “optionally substituted” it may be either unsubstituted or substituted. Likewise, when a group is described as being “substituted”, the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇ carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl) C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl) C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy) C₁-C₆ alkyl, —O(C₁-C₆ alkoxy) C₁-C₆ alkyl; (C₁-C₆ haloalkoxy) C₁-C₆ alkyl; —O(C₁-C₆ haloalkoxy) C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁₋₄ alkyl, monophosphate, diphosphate, triphosphate, and oxo (═O). Wherever a group is described as “optionally substituted” that group can be substituted with the above substituents.

As understood by one of ordinary skill in the art, a compound described herein may exist in ionized form, e.g., —CO₂⁻, —SO₃⁻ or —O—SO₃⁻. If a compound contains a positively or negatively charged substituent group, for example, —SO₃⁻, it may also contain a negatively or positively charged counterion such that the compound as a whole is neutral. In other aspects, the compound may exist in a salt form, where the counterion is provided by a conjugate acid or base.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

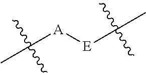

includes the substituent being oriented such that the A is attached at the leftmost attachment point of the molecule as well as the case in which A is attached at the rightmost attachment point of the molecule. In addition, if a group or substituent is depicted as

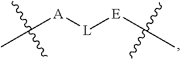

and L is defined an optionally present linker moiety; when L is not present (or absent), such group or substituent is equivalent to

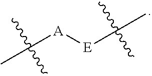

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxy group that is present in ribose. The nitrogen containing heterocyclic base can be purine or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N−1 of a pyrimidine or N−9 of a purine.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, adenine, guanine, deazapurine, 7-deaza adenine, 7-deaza guanine, hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” or “incorporating” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

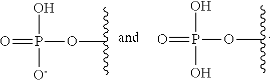 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably.

As used herein, the term “phasing” refers to a phenomenon in SBS that is caused by incomplete removal of the 3′ terminators and fluorophores, and failure to complete the incorporation of a portion of DNA strands within clusters by polymerases at a given sequencing cycle. Pre-phasing is caused by the incorporation of nucleotides without effective 3′ terminators, wherein the incorporation event goes 1 cycle ahead due to a termination failure. Phasing and pre-phasing cause the measured signal intensities for a specific cycle to consist of the signal from the current cycle as well as noise from the preceding and following cycles. As the number of cycles increases, the fraction of sequences per cluster affected by phasing and pre-phasing increases, hampering the identification of the correct base. Pre-phasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes. Accordingly, the discovery of nucleotide analogues which decrease the incidence of pre-phasing is surprising and provides a great advantage in SBS applications over existing nucleotide analogues. For example, the nucleotide analogues provided can result in faster SBS cycle time, lower phasing and pre-phasing values, and longer sequencing read lengths.

### Sequencing Methods Utilizing Palladium Catalysts

Some embodiments of the present disclosure relate to a method of determining the sequence of a target polynucleotide (e.g., single-stranded polynucleotide), comprising:

(a) contacting a copy polynucleotide/target polynucleotide complex with one or more different types of nucleotides (e.g., dATP, dCTP, dGTP, and dTTP or dUTP) in a first aqueous solution, wherein each of the nucleotides comprises a 3′ blocking group having the structure

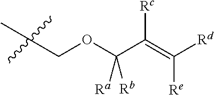

attached to the 3′ oxygen of the nucleotide, and wherein the copy polynucleotide is complementary to at least a portion of the target polynucleotide;

(b) incorporating one type of nucleotide into the copy polynucleotide to produce an extended copy polynucleotide;

(c) performing one or more fluorescent measurements to determine the identity of the incorporated nucleotide; and

(d) removing the 3′ blocking group of the incorporated nucleotide with a palladium catalyst;

wherein at least a portion of remaining palladium catalyst is inactivated by one or more palladium scavengers after step (d), wherein at least one palladium scavenger comprises one or more allyl moieties selected from the group consisting of —O-allyl, —S-allyl, —NR-allyl, and —N⁺RR′-allyl, and combinations thereof;

each of R^{a}, R^{b}, R^{c}, R^{d} and R^{e} is independently H, halogen, unsubstituted or substituted C₁-C₆ alkyl, or C₁-C₆ haloalkyl;

R is H, unsubstituted or substituted C₁-C₆ alkyl, unsubstituted or substituted C₂-C₆ alkenyl, unsubstituted or substituted C₂-C₆ alkynyl, unsubstituted or substituted C₆-C₁₀ aryl, unsubstituted or substituted 5 to 10 membered heteroaryl, unsubstituted or substituted C₃-C₁₀ carbocyclyl, or unsubstituted or substituted 5 to 10 membered heterocyclyl; and R′ is H, unsubstituted C₁-C₆ alkyl or substituted C₁-C₆ alkyl.

In some embodiments of the method described herein, the copy polynucleotide/target polynucleotide complex is formed by contacting the target polynucleotide with a single-stranded copy polynucleotide complementary to at least a portion of the target polynucleotide. In some embodiments, the incorporated nucleotide is a labeled nucleotide, the labeled nucleotide comprises a fluorescent label attached to the nucleotide optionally through a cleavable linker (e.g., the fluorescent label is attached to the nucleobase through a cleavable linker). In some such embodiments, step (d) also removes the fluorescent label. In some embodiments, the method further comprises step (e): washing the solid support with a second aqueous solution after the removal of the 3′ blocking group of the incorporated nucleotide. In some embodiments, the method further comprises repeating steps (a) through (d) or steps (a) through (e) until a sequence of at least a portion of the target polynucleotide strand is determined. In some embodiments, the cycle (i.e., steps (a) to (d) or steps (a) to (e)) is repeated at least 50 times, at least 100 times, at least 150 times, at least 200 times, at least 250 times, or at least 300 times. In some embodiments, the remaining palladium catalyst inactivated by one or more palladium scavengers is in the form Pd(II) and/or Pd(0) species. In some embodiments, the method is performed in parallel to determine a plurality of different polynucleotides (e.g., single-stranded polynucleotides).

Some further embodiments of the present disclosure relate to a method of determining the sequences of a plurality of target polynucleotides, comprising:

(a) contacting a solid support with sequencing primers under hybridization conditions, wherein the solid support comprises a plurality of different target polynucleotides immobilized thereon; and the sequencing primers are complementary to at least a portion of the target polynucleotides;

(b) contacting the solid support with a first aqueous solution comprising DNA polymerase and one or more of four different types of nucleotides (e.g., dATP, dCTP, dGTP, and dTTP or dUTP) under conditions suitable for DNA polymerase-mediated primer extension, wherein each of the nucleotides comprises a 3′ blocking group having the structure

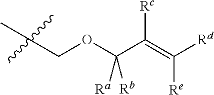

attached to the 3′ oxygen of the nucleotide;

(c) incorporating one type of nucleotides into the sequencing primers to produce extended copy polynucleotides;

(d) performing one or more fluorescent measurements of the extended copy polynucleotides; and

(e) removing the 3′ blocking group of the incorporated nucleotides with a palladium catalyst;

wherein at least a portion of remaining palladium catalyst is inactivated by one or more palladium scavengers after step (e), wherein at least one palladium scavenger comprises one or more allyl moieties selected from the group consisting of —O-allyl, —S-allyl, —NR-allyl, and —N⁺RR′-allyl, and combinations thereof;

each of R^{a}, R^{b}, R^{c}, R^{d} and R^{e} is independently H, halogen, unsubstituted or substituted C₁-C₆ alkyl, or C₁-C₆ haloalkyl;

R is H, unsubstituted or substituted C₁-C₆ alkyl, unsubstituted or substituted C₂-C₆ alkenyl, unsubstituted or substituted C₂-C₆ alkynyl, unsubstituted or substituted C₆-C₁₀ aryl, unsubstituted or substituted 5 to 10 membered heteroaryl, unsubstituted or substituted C₃-C₁₀ carbocyclyl, or unsubstituted or substituted 5 to 10 membered heterocyclyl; and

R′ is H, unsubstituted C₁-C₆ alkyl or substituted C₁-C₆ alkyl.

In some embodiments of the method described herein, one or more incorporated nucleotides is a labeled nucleotide, the labeled nucleotide comprises a detectable label (e.g., a fluorescent dye) attached to the nucleotide optionally through a cleavable linker (e.g., the detectable label is attached to the nucleobase through a cleavable linker). In some such embodiments, step (e) also removes the detectable label. In some embodiments, the method further comprises step (f): washing the solid support with a second aqueous solution after the removal of the 3′ blocking group of the incorporated nucleotides. In some embodiments, the method further comprises repeating steps (b) through (e) or steps (b) through (f) until sequences of at least a portion of the target polynucleotides are determined. In some embodiments, the cycle (i.e., steps (b) to (e) or steps (b) to (f)) is repeated at least 50 times, at least 100 times, at least 150 times, at least 200 times, at least 250 times, or at least 300 times. In some embodiments, the remaining palladium catalyst inactivated by the one or more palladium scavengers in the form Pd(II) and/or Pd(0) species.

In other embodiments of the method described herein, the incorporated nucleotide is unlabeled. One or more fluorescent labels may be introduced after incorporation by using labeled affinity reagents containing one or more fluorescent dyes. For example, one, two, three or each of the four different types of nucleotides (e.g., dATP, dCTP, dGTP and dTTP or dUTP) in the first aqueous solution may be unlabeled. Each of the four types of nucleotides (e.g., dNTPs) has a 3′ hydroxy blocking group described herein to ensure that only a single base can be added by a polymerase to the 3′ end of the copy polynucleotide. After incorporation of an unlabeled nucleotide, an affinity reagent is then introduced that specifically binds to the incorporated dNTP to provide a labeled extension product comprising the incorporated dNTP. Uses of unlabeled nucleotides and affinity reagents in sequencing by synthesis have been disclosed in U.S. Publication No. 2013/0079232. A modified sequencing method of the present disclosure using unlabeled nucleotides may include the following steps:

(a′) contacting a copy polynucleotide/target polynucleotide complex with one or more unlabeled nucleotides (e.g., dATP, dCTP, dGTP, and dTTP or dUTP) in first aqueous solution, wherein each of the nucleotides comprises a 3′ blocking group having the structure

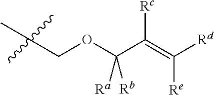

attached to the 3′ oxygen of the nucleotide (each of R^{a}, R^{b}, R^{c}, R^{d} and R^{e} is defined above), and wherein the copy polynucleotide is complementary to at least a portion of the target polynucleotide;

(b′-1) incorporating one type of nucleotide into the copy polynucleotide to produce an extended copy polynucleotide;

(b′-2) contacting the extended copy polynucleotide with a set of affinity reagents under conditions wherein one affinity reagent binds specifically to the incorporated unlabeled nucleotide to provide a labeled extended copy polynucleotide/target polynucleotide complex;

(c′) performing one or more fluorescent measurements of the labeled extended copy polynucleotide/target polynucleotide complex to determine the identity of the incorporated nucleotide; and

(d′) removing the 3′ blocking group of the incorporated nucleotide with a palladium catalyst;

wherein at least a portion of remaining palladium catalyst is inactivated by one or more palladium scavengers after step (d′), wherein at least one palladium scavenger comprises one or more allyl moieties selected from the group consisting of —O-allyl, —S-allyl, —NR-allyl, and —N⁺RR′-allyl, and combinations thereof.

In some embodiments of the method described herein, the copy polynucleotide/target polynucleotide complex is formed by contacting the target polynucleotide with a single-stranded copy polynucleotide complementary to at least a portion of the target polynucleotide. The affinity reagents may include small molecules or protein tags that may bind to a hapten moiety of the nucleotide (such as streptavidin-biotin, anti-DIG and DIG, anti-DNP and DNP), antibody (including but not limited to binding fragments of antibodies, single chain antibodies, bispecific antibodies, and the like), aptamers, knottins, affimers, or any other known agent that binds an incorporated nucleotide with a suitable specificity and affinity. In further embodiments, one affinity reagent may be labeled with multiple copies of the same fluorescent dyes. In some embodiments, the Pd catalyst also removes the labeled affinity reagent. For example, the hapten moiety of the unlabeled nucleotide may be attached to the nucleobase through a cleavable linker, which may be cleaved by the Pd catalyst. In some embodiments, the method further comprises repeating steps (a′) through (d′) until a sequence of at least a portion of the target polynucleotide strand is determined. In some embodiments, the cycle (i.e., steps (a′) through (d′)) is repeated at least 50 times, at least 100 times, at least 150 times, at least 200 times, at least 250 times, or at least 300 times. In some embodiments of the method described herein, the method further comprises: (e′) washing the removed a 3′ blocking group away from the copy polynucleotide/target polynucleotide complex by using a second aqueous solution. In some embodiments, the method further comprises repeating steps (a′) through (e′) until a sequence of at least a portion of the target polynucleotide strand is determined. In some embodiments, the cycle (i.e., steps (a′) through (e′) is repeated at least 50 times, at least 100 times, at least 150 times, at least 200 times, at least 250 times, or at least 300 times. In some embodiments, the remaining palladium catalyst inactivated by the one or more palladium scavengers in the form of Pd(II) and/or Pd(0) species. In some embodiments, this modified method is performed in parallel to determine a plurality of different polynucleotides (e.g., single-stranded polynucleotides).

In some embodiments of any of the methods described herein, the palladium scavenger comprises one or more allyl moieties is in the first aqueous solution. In some instances, the first aqueous solution is also known as the incorporation mix (IMX). In some such embodiments, such palladium scavenger is compatible with the other sequencing reagents in the first aqueous solution, which may also include a polymerase (such as DNA polymerase), in addition to the one or more different types of nucleotides. In some such embodiments, the polymerase is a DNA polymerase, such as a mutant of 9° N polymerase (e.g., those disclosed in WO 2005/024010, which is incorporated by reference), for example, Pol 812, Pol 1901, Pol 1558 or Pol 963. The amino acid sequences of Pol 812, Pol 1901, Pol 1558 or Pol 963 DNA polymerases are described, for example, in U.S. Patent Publication Nos. 2020/0131484 A1 and 2020/0181587 A1, both of which are incorporated by reference herein. In some embodiments, the first aqueous solution further comprises one or more buffering agents. The buffering agents may comprise a primary amine, a secondary amine, a tertiary amine, a natural amino acid, or a non-natural amino acid, or combinations thereof. In further embodiments, the buffering agents comprise ethanolamine or glycine, or a combination thereof. In one embodiment, the buffer agent comprises or is glycine. In further embodiments, the palladium scavenger comprises one or more allyl moieties does not require a separate washing step prior to the next incorporation cycle. In further embodiments, the palladium scavenger in the first aqueous solution is a Pd(0) scavenger described herein. In some embodiments, the Pd(0) scavenger is premixed with the DNA polymerase and/or the one or more of four types of nucleotides (e.g., dATP, dCTP, dGTP, and dTTP or dUTP). In other embodiments, the Pd(0) scavenger is stored separately form the DNA polymerase and/or the one or more of four types of nucleotides and is mixed with these components shortly before sequencing run starts.

In some embodiments of any of the methods described herein, the concentration of the palladium scavenger comprising one or more allyl moieties (e.g., the Pd(0) scavenger) in the first aqueous solution is from about 0.1 mM to about 100 mM, from 0.2 mM to about 75 mM, from about 0.5 mM to about 50 mM, from about 1 mM to about 20 mM, or from about 2 mM to about 10 mM. In further embodiments, the concentration of the palladium scavenger (e.g., the Pd(0) scavenger) is about 0.1 mM, 0.2 mM, 0.3, mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 12.5 mM, 15 mM, 17.5 mM or 20 mM, or a range defined by any two of the preceding values. In one embodiment, the Pd scavenger is Compound B in a concentration of about 2 mM. In other embodiment, the Pd scavenger is Compound O in a concentration of about 0.5 mM. In further embodiments, the concentration of such palladium scavenger is the concentration in the first aqueous solution. In further embodiments, the pH of the first aqueous solution is about 9.

In some other embodiments of any of the methods described herein, the palladium scavenger comprises one or more allyl moieties is in a solution when performing one or more fluorescent measurements. In such embodiment, such palladium scavenger is compatible with the sequencing reagents of the scanning solution (also known as the scan mix). In further embodiments, the one or more palladium scavengers does not require a separate washing step prior to the next incorporation cycle. In further embodiments, the palladium scavenger in the scan solution is a Pd(0) scavenger described herein.

In other embodiments of the methods described herein, the palladium scavenger comprises one or more allyl moieties is in the post cleavage wash solution (i.e., the second aqueous solution). In further embodiments, the palladium scavenger in the post cleavage wash solution is a Pd(0) scavenger described herein. In some such embodiment, the post cleavage wash solution does not comprise lipoic acid or 3,3′-dithiodipropionic acid (DDPA).

In still other embodiments of the method described herein, the palladium scavenger comprises one or more allyl moieties may be present both in the first aqueous solution (e.g., incorporation mix) and in the second aqueous solution (e.g., post cleavage wash solution), or present in both the first aqueous solution and the scan mix. In some such embodiment, the post cleavage wash solution does not comprise lipoic acid or DDPA.

In some embodiments of the methods described herein, the palladium scavenger comprising one or more —O-allyl moieties has the structure:

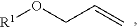

wherein R¹ is C₁-C₁₂ alkyl optionally substituted with one or more R^{x}, C₂-C₁₂ alkenyl optionally substituted with one or more R^{x}, C₂-C₁₂ alkynyl optionally substituted with one or more R^{x}, unsubstituted amino, substituted amino, C₆-C₁₀ aryl, (C₆-C₁₀ aryl) C₁-C₆ alkyl, 5 to 10 membered heteroaryl, (5 to 10 membered heteroaryl) C₁-C₆ alkyl, C₃-C₁₀ carbocyclyl, (C₃-C₁₀ carbocyclyl) C₁-C₆ alkyl, 3 to 10 membered heterocyclyl, (3 to 10 membered heterocyclyl) C₁-C₆ alkyl, a monosaccharide moiety, a disaccharide moiety, an oligosaccharide moiety, an amino acid moiety, —C(═O)NR^{f1}R^{g1}, —P(═O)OR^{f1}OR^{g1}, —C(═O)R^{h1}, —C(═O)OR^{h1} or —S(═O)₂R^{j1}, wherein each of C₆-C₁₀ aryl, 5 to 10 membered heteroaryl, C₃-C₁₀ carbocyclyl and 3 to 10 membered heterocyclyl is optionally substituted with one or more R^{x};
each of R^{f1} and R^{g1} is independently H, C₁-C₆ alkyl optionally substituted with one or more R^{x}, C₆-C₁₀ aryl optionally substituted with one or more R^{x}, or 5 to 10 membered heteroaryl optionally substituted with one or more R^{x};
each R^{h1} is independently C₁-C₆ alkyl optionally substituted with one or more R^{x}, C₆-C₁₀ aryl optionally substituted with one or more R^{x}, or 5 to 10 membered heteroaryl optionally substituted with one or more R^{x};
each R^{j1} is independently hydroxy, C₁-C₆ alkyl optionally substituted with one or more R^{x}, C₆-C₁₀ aryl optionally substituted with one or more R^{x}, or 5 to 10 membered heteroaryl optionally substituted with one or more R^{x}; and
each R^{x} is independently amino, halo, hydroxy, carboxy, cyano, (C₁-C₆ alkyl)amino, C-amido, N-amido, unsubstituted and substituted C₁-C₆ alkyl, C₁-C₆ haloalkyl, unsubstituted and substituted C₁-C₆ alkoxy, C₁-C₆ haloalkoxy, unsubstituted and substituted C₆-C₁₀ aryloxy, sulfo, sulfonate, or —O—CH₂—CH═CH₂.

In some embodiments, R¹ is C₁-C₆ alkyl optionally substituted with one or more R^{x}, C₂-C₆ alkenyl optionally substituted with one or more R^{x}, unsubstituted amino, substituted amino, C₆-C₁₀ aryl optionally substituted with one or more R^{x}, 5 to 10 membered heteroaryl optionally substituted with one or more R^{x}, a monosaccharide moiety, a disaccharide moiety, an amino acid moiety, —C(═O)NH₂, —P(═O)(OH)₂, or —S(═O)₂OH, and wherein each R^{x} is independently amino, cyano, halo, hydroxy, carboxy, unsubstituted and substituted C₁-C₆ alkyl, C₁-C₆ haloalkyl, unsubstituted and substituted C₁-C₆ alkoxy, unsubstituted and substituted C₆-C₁₀ aryloxy, or —O—CH₂—CH═CH₂. In some such embodiments, R¹ is a monosaccharide moiety with five or six membered sugar ring, or a modified analog thereof (e.g., glucopyranoside). In some such embodiments, R¹ is a C₁-C₆ alkyl unsubstituted or substituted with one or more R^{x}, where R^{x} is independently hydroxy, carboxy, substituted C₁-C₆ alkoxy, substituted C₆-C₁₀ aryloxy (e.g., —OPh) and —O—CH₂—CH═CH₂. In some such embodiment, R¹ is C₂-C₆ alkenyl (e.g., C₃ alkenyl). In some such embodiments, R¹ is an amino acid moiety where the amino moiety may be further protected (e.g., R¹ is a N-Boc-protected tyrosine residue). When R¹ is an amino acid moiety, it also includes any derivative or analogs of the amino acid moiety. For example, the free amino group of the amino acid residue may be protected with an amino protecting group (e.g., a tert-butyloxycarbonyl or Boc protecting group). The carboxy group of the amino acid residue may be in the form of an ester. In one embodiment, R¹ is an amino group. In some other embodiments, R¹ is a five, six, nine or ten membered heteroaryl or heterocyclyl comprising one, two, three or four heteroatoms selected from O, S and N optionally substituted with one or more R^{x} (where R^{x} is independently hydroxy, carboxy, and —O—CH₂—CH═CH₂). In some other embodiments, R¹ is phenyl optionally substituted with one or more R^{x} (where R^{x} is independently hydroxy, carboxy, and —O—CH₂—CH═CH₂). When R¹ comprises a phosphate, sulfo or sulfonate, one or more hydroxy group could be in the anionic form and the palladium scavenger may also comprises one or more cations such that the scavenger is in a salt form and does not bear any charges. In any embodiments of the Pd scavenger comprising one or more —O-allyl moieties, one or more hydrogen atoms of the allyl moiety may also be substituted (e.g., with halogen, C₁-C₆ alkyl, or C₁-C₆ haloalkyl).

Non-limiting examples of the palladium scavenger comprising one or more —O-allyl or allyl moieties include the following:

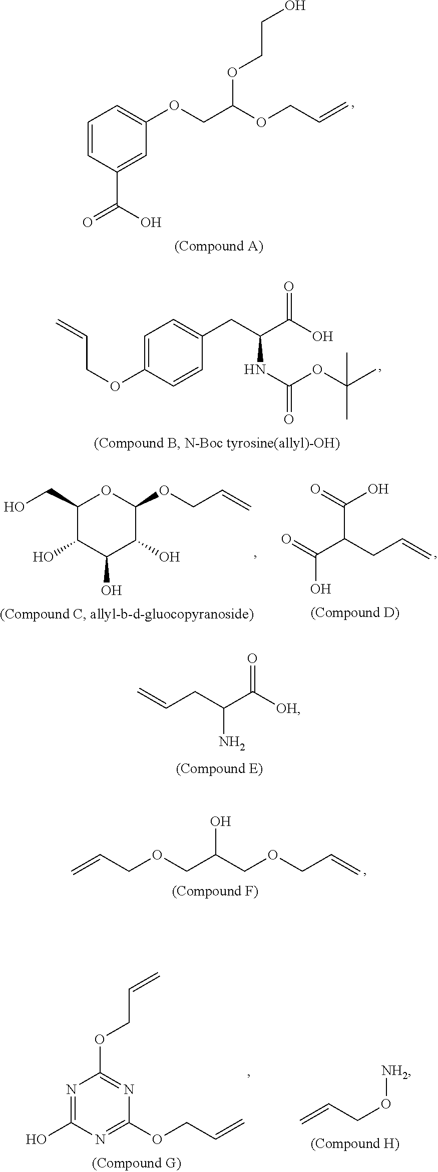

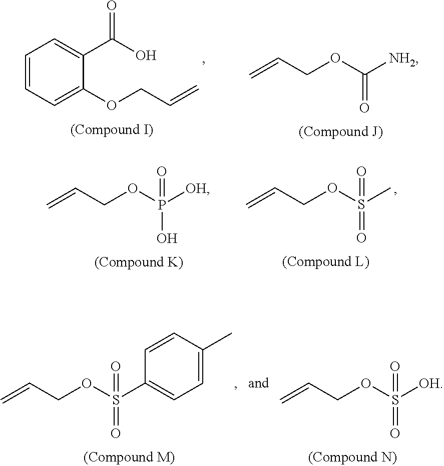

In some embodiments of the methods described herein, the palladium scavenger comprising one or more —S-allyl moieties has the structure:

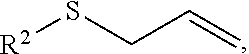

wherein R² is C₁-C₁₂ alkyl optionally substituted with one or more R^{y}, C₂-C₁₂ alkenyl optionally substituted with one or more R^{y}, C₂-C₁₂ alkynyl optionally substituted with one or more R^{y}, unsubstituted amino, substituted amino, C₆-C₁₀ aryl, (C₆-C₁₀ aryl) C₁-C₆ alkyl, 5 to 10 membered heteroaryl, (5 to 10 membered heteroaryl) C₁-C₆ alkyl, C₃-C₁₀ carbocyclyl, (C₃-C₁₀ carbocyclyl) C₁-C₆ alkyl, 3 to 10 membered heterocyclyl, (3 to 10 membered heterocyclyl) C₁-C₆ alkyl, a monosaccharide moiety, a disaccharide moiety, an oligosaccharide moiety, an amino acid moiety, —C(═O)NR^{f2}R^{g2}, —P(═O)OR^{f2}QR^{g2}, —C(═O)R^{h2}, —C(═O)OR^{h2} or —S(═O)₂R^{j2}, wherein each of C₆-C₁₀ aryl, 5 to 10 membered heteroaryl, C₃-C₁₀ carbocyclyl and 3 to 10 membered heterocyclyl is optionally substituted with one or more R^{y};
each of R^{f2} and R^{g2} is independently H, C₁-C₆ alkyl optionally substituted with one or more R^{y}, C₆-C₁₀ aryl optionally substituted with one or more R^{y}, or 5 to 10 membered heteroaryl optionally substituted with one or more R^{y};
each R^{h2} is independently C₁-C₆ alkyl optionally substituted with one or more R^{y}, C₆-C₁₀ aryl optionally substituted with one or more R^{y}, or 5 to 10 membered heteroaryl optionally substituted with one or more R^{y};
each R^{j2} is independently hydroxy, C₁-C₆ alkyl optionally substituted with one or more R^{y}, C₆-C₁₀ aryl optionally substituted with one or more R^{y}, or 5 to 10 membered heteroaryl optionally substituted with one or more R^{y}; and each R^{y} is independently amino, halo, hydroxy, carboxy, cyano, (C₁-C₆ alkyl)amino, C-amido, N-amido, unsubstituted and substituted C₁-C₆ alkyl, C₁-C₆ haloalkyl, unsubstituted and substituted C₁-C₆ alkoxy, C₁-C₆ haloalkoxy, unsubstituted and substituted C₆-C₁₀ aryloxy, sulfo, sulfonate, or —S—CH₂—CH═CH₂.

In some embodiments, R² is C₁-C₆ alkyl optionally substituted with one or more R^{y}, C₂-C₆ alkenyl optionally substituted with one or more R^{y}, unsubstituted amino, substituted amino, C₆-C₁₀ aryl optionally substituted with one or more R^{y}, 5 to 10 membered heteroaryl optionally substituted with one or more R^{y}, a monosaccharide moiety, a disaccharide moiety, an amino acid moiety, —C(═O)NH₂, —P(═O)(OH)₂, or —S(═O)₂OH, and wherein each R^{y} is independently amino, cyano, halo, hydroxy, carboxy, unsubstituted and substituted C₁-C₆ alkyl, C₁-C₆ haloalkyl, unsubstituted and substituted C₁-C₆ alkoxy, unsubstituted and substituted C₆-C₁₀ aryloxy, or —S—CH₂—CH═CH₂. In some such embodiments, R² is a monosaccharide moiety with five or six membered sugar ring, or a modified analog thereof (e.g., glucopyranoside). In some such embodiments, R² is a C₁-C₆ alkyl unsubstituted or substituted with one or more R^{y}, where R^{y} is independently hydroxy, carboxy, substituted C₁-C₆ alkoxy, substituted C₆-C₁₀ aryloxy (e.g., —OPh) and —S—CH₂—CH═CH₂. In some such embodiment, R² is C₂-C₆ alkenyl (e.g., C₃ alkenyl). In some such embodiments, R² is an amino acid moiety where the amino moiety may be further protected (e.g., R² is a N-Boc-protected tyrosine residue). When R² is an amino acid moiety, it also includes any derivative or analogs of the amino acid moiety. For example, the free amino group of the amino acid moiety may be protected with an amino protecting group (e.g., a tert-butyloxycarbonyl or Boc protecting group). The carboxy group of the amino acid moiety may be in the form of an ester. In one embodiment, R² is an amino group. In some other embodiments, R² is a five, six, nine or ten membered heterocyclyl or heteroaryl comprising one, two, three or four heteroatoms selected from O, S and N optionally substituted with one or more R^{y} (where R^{y} is independently hydroxy, carboxy, and —S—CH₂—CH═CH₂). In some other embodiments, R² is phenyl optionally substituted with one or more R^{y} (where R^{y} is independently hydroxy, carboxy, and —S—CH₂—CH═CH₂). When R² comprises a phosphate, sulfo or sulfonate, one or more hydroxy group could be in the anionic form and the palladium scavenger may also comprises one or more cations such that the scavenger is in a salt form and does not bear any charges. In any embodiments of the Pd scavenger comprising one or more —S-allyl moieties, one or more hydrogen atoms of the allyl moiety may also be substituted (e.g., with halogen, C₁-C₆ alkyl, or C₁-C₆ haloalkyl).

Non-limiting examples of the palladium scavenger comprising one or more —S-allyl moieties include the following:

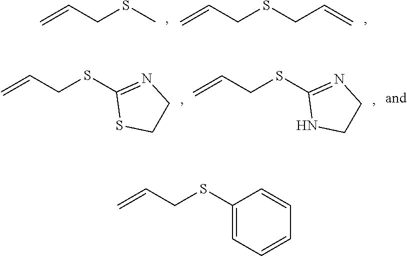

In some embodiments of the methods described herein, the palladium scavenger comprising one or more —NR-allyl or —N⁺RR′-allyl moieties having the structure:

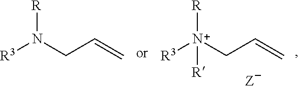

wherein Z is an anion;
each R³ is independently C₁-C₁₂ alkyl optionally substituted with one or more R^{z}, C₂-C₁₂ alkenyl optionally substituted with one or more R^{z}, C₂-C₁₂ alkynyl optionally substituted with one or more R^{z}, unsubstituted amino, substituted amino, C₆-C₁₀ aryl, (C₆-C₁₀ aryl) C₁-C₆ alkyl, 5 to 10 membered heteroaryl, (5 to 10 membered heteroaryl) C₁-C₆ alkyl, C₃-C₁₀ carbocyclyl, (C₃-C₁₀ carbocyclyl) C₁-C₆ alkyl, 3 to 10 membered heterocyclyl, (3 to 10 membered heterocyclyl) C₁-C₆ alkyl, a monosaccharide moiety, a disaccharide moiety, an oligosaccharide moiety, an amino acid moiety, —C(═O)NR^{f3}R^{g3}, —P(═O)OR^{f3}OR^{g3}, —C(═O)R^{h3}, —C(═O)OR^{h3} or —S(═O)₂R^{j3}, wherein each of C₆-C₁₀ aryl, 5 to 10 membered heteroaryl, C₃-C₁₀ carbocyclyl and 3 to 10 membered heterocyclyl is optionally substituted with one or more R^{z};
each of R^{f3} and R^{g3} is independently H, C₁-C₆ alkyl optionally substituted with one or more R^{z}, C₆-C₁₀ aryl optionally substituted with one or more R^{z}, or 5 to 10 membered heteroaryl optionally substituted with one or more R^{z};
each R^{h3} is independently C₁-C₆ alkyl optionally substituted with one or more R^{z}, C₆-C₁₀ aryl optionally substituted with one or more R^{z}, or 5 to 10 membered heteroaryl optionally substituted with one or more R^{z};
each R^{j3} is independently hydroxy, C₁-C₆ alkyl optionally substituted with one or more R^{z}, C₆-C₁₀ aryl optionally substituted with one or more R^{z}, or 5 to 10 membered heteroaryl optionally substituted with one or more R^{z}; and
each R^{z} is independently amino, halo, hydroxy, carboxy, cyano, (C₁-C₆ alkyl)amino, C-amido, N-amido, unsubstituted and substituted C₁-C₆ alkyl, C₁-C₆ haloalkyl, unsubstituted and substituted C₁-C₆ alkoxy, C₁-C₆ haloalkoxy, unsubstituted and substituted C₆-C₁₀ aryloxy, sulfo, sulfonate, or —NH—CH₂—CH═CH₂.

In some embodiments, R is H or C₁-C₆ alkyl. In some embodiments, R′ is H or C₁-C₆ alkyl. In some further embodiments, R³ is a C₁-C₆ alkyl optionally substituted with one or more R^{z}, C₂-C₆ alkenyl optionally substituted with one or more R^{z}, unsubstituted amino, substituted amino, C₆-C₁₀ aryl optionally substituted with one or more R^{z}, 5 to 10 membered heteroaryl optionally substituted with one or more R^{z}, a monosaccharide moiety, a disaccharide moiety, an amino acid moiety, —C(═O)NH₂, —P(═O)(OH)₂, or —S(═O)₂OH, and wherein each R^{z} is independently amino, cyano, halo, hydroxy, carboxy, unsubstituted and substituted C₁-C₆ alkyl, C₁-C₆ haloalkyl, unsubstituted and substituted C₁-C₆ alkoxy, unsubstituted and substituted C₆-C₁₀ aryloxy, or —NH—CH₂—CH═CH₂. In some such embodiments, R³ is a monosaccharide moiety with five or six membered sugar ring, or a modified analog thereof (e.g., glucopyranoside). In some such embodiments, R³ is a C₁-C₆ alkyl substituted with one or more R^{z}, where R^{z} is independently hydroxy, carboxy, substituted C₁-C₆ alkoxy, substituted C₆-C₁₀ aryloxy (e.g., —OPh) and —NH—CH₂—CH═CH₂. In some such embodiment, R³ is C₂-C₆ alkenyl (e.g., C₃ alkenyl). In some such embodiments, R³ is an amino acid moiety where the amino moiety may be further protected (e.g., R³ is a N-Boc-protected tyrosine residue). When R³ is an amino acid residue, it also includes any derivative or analogs of the amino acid moiety. For example, the free amino group of the amino acid moiety may be protected with an amino protecting group (e.g., a tert-butyloxycarbonyl or Boc protecting group). The carboxy group of the amino acid moiety may be in the form of an ester. In one embodiment, R³ is an amino group. In some other embodiments, R³ is a five, six, nine or ten membered heterocyclyl or heteroaryl comprising one, two, three or four heteroatoms selected from O, S and N optionally substituted with one or more R^{z} (where R^{z} is independently hydroxy, carboxy, and —NH—CH₂—CH═CH₂). In some other embodiments, R³ is phenyl optionally substituted with one or more R^{z} (where R^{z} is independently hydroxy, carboxy, and —NH—CH₂—CH═CH₂). When R³ comprises a phosphate, sulfo or sulfonate, one or more hydroxy group could be in the anionic form and the palladium scavenger may also comprises one or more cations such that the scavenger is in a salt form and does not bear any charges. In any embodiments of the Pd scavenger comprising one or more —NR-allyl or —N⁺RR′-allyl moieties, one or more hydrogen atoms of the allyl moiety may also be substituted (e.g., with halogen, C₁-C₆ alkyl, or C₁-C₆ haloalkyl).

Non-limiting examples of the palladium scavenger comprising one or more —NR-allyl or —N⁺RR′-allyl moieties include the following:

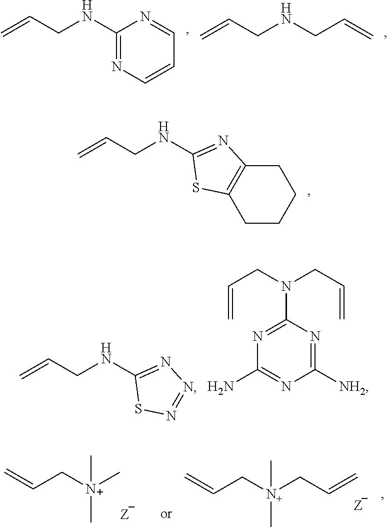

where Z⁻ is an anion (e.g., a halide anion such as F⁻ or Cl⁻). In one embodiment, the kit comprises the palladium scavenger

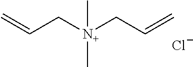

(Compound O, diallyldimethylammonium chloride, also known as DADMAC).

In any embodiments of the Pd scavengers comprising one or more allyl moieties (e.g., —O-allyl, —S-allyl, —NR-allyl or —N⁺RR′-allyl), such Pd scavenger is a Pd(0) scavenger.

Palladium Catalysts

In some embodiments, the Pd catalyst used for removing or cleaving the 3′ blocking group described herein is water soluble. In some such embodiments, the Pd catalyst is the active Pd(0) form. In some instances, the Pd(0) catalyst may be generated in situ from reduction of a Pd complex or Pd precatalyst (e.g., a Pd(II) complex) by reagents such as alkenes, alcohols, amines, phosphines, or metal hydrides. Suitable palladium sources include Pd(CH₃CN)₂Cl₂, [PdCl(Allyl)]₂, [Pd(Allyl)(THP)]Cl, [Pd(Allyl)(THP)₂]Cl, Pd(OAc)₂, Pd(PPh₃)₄, Pd(dba)₂, Pd(Acac)₂, PdCl₂(COD), and Pd(TFA)₂. In one such embodiment, the Pd(0) complex is generated in situ from an organic or inorganic salt of palladate (II), for example, Na₂PdCl₄ or K₂PdCl₄. In another embodiment, the palladium source is allyl Pd(II) chloride dimer [(Allyl)PdCl]₂ or [PdCl(C₃H₅)]₂. In some embodiments, the Pd(0) catalyst is generated in an aqueous solution by mixing a Pd(II) complex with a water soluble phosphine. Suitable phosphines include water soluble phosphines, such as tris(hydroxypropyl)phosphine (THP), tris(hydroxymethyl)phosphine (THMP), 1,3,5-triaza-7-phosphaadamantane (PTA), bis(p-sulfonatophenyl)phenylphosphine dihydrate potassium salt, tris(carboxyethyl)phosphine (TCEP), and triphenylphosphine-3,3′,3″-trisulfonic acid trisodium salt, or combinations thereof.

In some embodiments, the palladium catalyst is prepared by mixing [(Allyl)PdCl]₂ with THP in situ. The molar ratio of [(Allyl)PdCl]₂ and the THP may be about 1:2, 1:3, 1:4, 1:5, 1:6, 1:7, 1:8, 1:9, or 1:10. In one embodiment, the molar ratio of [(Allyl)PdCl]₂ to THP is 1:10. In some other embodiment, the palladium catalyst is prepared by mixing a water soluble Pd reagent such as Na₂PdCl₄ or K₂PdCl₄ with THP in situ. The molar ratio of Na₂PdCl₄ or K₂PdCl₄ and THP may be about 1:2, 1:3, 1:4, 1:5, 1:6, 1:7, 1:8, 1:9, or 1:10. In one embodiment, the molar ratio of Na₂PdCl₄ or K₂PdCl₄ to THP is about 1:3. In another embodiment, the molar ratio of Na₂PdCl₄ or K₂PdCl₄ to THP is about 1:3.5.

The Pd complex and the water-soluble phosphine for use in the cleavage step of the method described herein may be in a composition or a mixture, also called cleavage mix. In some further embodiments, the cleavage mix may contain additional buffer reagents, such as a primary amine, a secondary amine, a tertiary amine, a natural amino acid, a non-natural amino acid, a carbonate salt, a phosphate salt, or a borate salt, or combinations thereof. In some further embodiments, the buffer reagent comprises ethanolamine (EA), tris(hydroxymethyl)aminomethane (Tris), glycine, sodium carbonate, sodium phosphate, sodium borate, dimethylethanolamine (DMEA), diethylethanolamine (DEEA), N,N,N′,N′-tetramethylethylenediamine (TMEDA), or N,N,N′,N′-tetraethylethylenediamine (TEEDA), or combinations thereof. In one embodiment, the one or more buffer reagents comprise DEEA. In another embodiment, the one or more buffer reagents contains one or more inorganic salts such as a carbonate salt, a phosphate salt, or a borate salt, or combinations thereof. In one embodiment, the inorganic salt is a sodium salt.

In some embodiments, the molar ratio of the palladium catalyst to the palladium scavenger comprising one or more allyl moieties is about 1:100, 1:50, 1:20, 1:10 or 1:5. In some further embodiments, the palladium scavenger comprises one or more allyl moieties is a palladium scavenger for Pd(0), the active form of the Pd catalyst.

In some embodiments, the cleavage condition for the 3′ blocking group is the same as the condition for cleaving the cleavable linker of the nucleotide. For example, the nucleotide may comprise a linker moiety that is the same as the 3′ blocking group. In other embodiments, the cleavage condition for the 3′ blocking group is different from the condition for cleaving the cleavable linker of the nucleotide.

Additional Palladium Scavengers

In some embodiments of the methods described herein, the method may further use additional palladium scavenger(s), such as Pd(II) scavenger(s). In some such embodiments, the use of additional Pd scavenger(s) may improve the phasing value of the sequencing metrics. For example, the additional Pd scavenger(s) may comprise an isocyanoacetate (ICNA) salt, ethyl isocyanoacetate, methyl isocyanoacetate, cysteine (e.g., L-cysteine) or a salt thereof (e.g., N-acetyl-L-cysteine), potassium ethylxanthogenate, potassium isopropyl xanthate, glutathione, ethylenediaminetetraacetic acid (EDTA), iminodiacetic acid, nitrilodiacetic acid, trimercapto-S-triazine, dimethyldithiocarbamate, dithiothreitol, mercaptoethanol, allyl alcohol, propargyl alcohol, thiol, thiosulfate salt (e.g., sodium thiosulfate or potassium thiosulfate), tertiary amine and/or tertiary phosphine, or combinations thereof. In one embodiment, the method also includes the use of L-cysteine or a salt thereof. In another embodiment, the method also includes the use of a thiosulfate salt such as sodium thiosulfate (Na₂S₂O₃). In some embodiments, the additional Pd scavenger is a scavenger for Pd(II). In some such embodiments, the Pd(II) scavenger (e.g., L-cysteine or sodium thiosulfate) is in the first aqueous solution. In other embodiments, the Pd(II) scavenger (e.g., L-cysteine or sodium thiosulfate) is in the post cleavage wash solution (i.e., the second aqueous solution). In other embodiments, the Pd(II) scavenger (e.g., L-cysteine or sodium thiosulfate) may be present both in the first aqueous solution and the second aqueous solution. In other embodiments, the Pd(II) scavenger (e.g., L-cysteine or sodium thiosulfate) may be present in the scan mixture (i.e., the solution in which one or more fluorescent measurements of the incorporated nucleotide are performed). In other embodiments, the Pd(II) scavenger may be present in one or more of incorporation mixture (e.g., the first aqueous solution), the scan mixture, or the post-cleavage wash solution (e.g., the second aqueous solution). In further embodiments, the concentration of the Pd(II) scavenger such as L-cysteine or sodium thiosulfate in the first aqueous solution or the second aqueous solution is from about 0.1 mM to about 100 mM, from 0.2 mM to about 75 mM, from about 0.5 mM to about 50 mM, from about 1 mM to about 20 mM, or from about 2 mM to about 10 mM. In further embodiments, the concentration of the Pd(II) scavenger such as L-cysteine or sodium thiosulfate is about 0.1 mM, 0.5 mM, 1 mM, 2 mM, 3 mM, 4 mM, 5 mM, 6 mM, 6.5 mM, 7 mM, 8 mM, 9 mM, 10 mM, 12.5 mM, 15 mM, 17.5 mM or 20 mM, or a range defined by any two of the preceding values. In further embodiments, the Pd(II) scavenger is in the second aqueous solution, and the concentration of the Pd(II) scavenger in the second aqueous solution is about 10 mM.

In some embodiments of the methods described herein, all Pd scavengers are in the first aqueous solution. In some other embodiments of the methods described herein, all Pd scavengers are in the second aqueous solution. In some other embodiments, the one or more Pd scavenger comprising one or more allyl moieties (e.g., Pd(0) scavenger) is in the incorporation mixture (i.e., first aqueous solution), and the Pd(II) scavenger(s) is in the post cleavage wash solution (i.e., second aqueous solution). In further embodiment, the post cleavage wash solution does not contain lipoic acid or DDPA. In other embodiments, the method does not include a post-cleavage wash step.

In some embodiments of the methods described herein, the use of one or more Pd scavenger comprising one or more allyl moieties (e.g., Pd(0) scavenger) reduces the prephasing values of the sequencing run to less than about 0.1%, 0.09%, 0.08%, 0.07%, 0.06%, 0.05%, 0.04%, 0.03%, 0.02% or 0.01%. In some embodiments, the prephasing value refers to the value measured after 50 cycles, 75 cycles, 100 cycles, 125 cycles, 150 cycles, 200 cycles, 250 cycles or 300 cycles.

In some embodiments of the methods described herein, the use of one or more Pd(II) scavengers reduces the phasing values of the sequencing run to less than about 0.5%, 0.4%, 0.3%, 0.2%, 0.1%, 0.09%, 0.08%, 0.07%, 0.06%, or 0.05%. In some embodiments, the phasing value refers to the value measured after 50 cycles, 75 cycles, 100 cycles, 125 cycles, 150 cycles, 200 cycles, 250 cycles or 300 cycles.

In some embodiments of the methods described herein, the target polynucleotide is immobilized to a surface of a substrate. In some further embodiments, the surface comprises a plurality of immobilized target polynucleotides, for example, an array of different immobilized target polynucleotides. In some such embodiments, the substrate comprises glass, modified or functionalized glass, plastics, polysaccharides, nylon, nitrocellulose, resins, silica, silicon, modified silicon, carbon, metals, inorganic glasses, or optical fiber bundles, or combinations thereof. In some further embodiments, the substrate is a flowcell, a nanoparticle, or a bead (such as spherical silica beads, inorganic nanoparticles, magnetic nanoparticles, cadmium-based dots, and cadmium free dots, or a bead disclosed in U.S. Publication No. 2021/0187470 A1, which is incorporated by reference). In one embodiment, the substrate is a flowcell comprising patterned nanowells separated by interstitial regions, and wherein the immobilized target polynucleotides reside inside the patterned nanowells.

In some embodiments of any of the methods described herein, the method is performed on an automated sequencing instrument, and wherein the automated sequencing instrument comprises two light sources operating at different wavelengths (e.g., at about 450 nm to about 460 nm, and about 520 nm to about 540 nm, in particular at about 460 nm and about 532 nm). In other embodiments, the automated sequencing instrument comprises a single light source operating at one wavelength.

In any embodiments of the methods described herein, the one or more palladium scavengers does not include lipoic acid or 3,3′-dithiodipropionic acid (DDPA).

In any embodiments of the method described herein, one skilled in the art should understand that the palladium scavenger may not completely inactivate the residual/remaining Pd catalyst (in the form of Pd(0) and/or Pd(II) species) after the cleavage step and there may be a trace amount of Pd(0) or Pd(II) species remaining. As a result, the prephasing and phasing values might not be reduced to zero.

Nucleotides with 3′ Blocking Groups

Some embodiments of the present disclosure relate to a nucleotide molecule comprising a nucleobase, a ribose or deoxyribose moiety, and a 3′ hydroxy blocking group comprising an allyl moiety, such as a 3′ blocking group having the structure

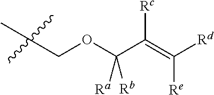

attached to the 3′ oxygen of the nucleotide, wherein each of R^{a}, R^{b}, R^{c}, R^{d} and R^{e} is independently H, halogen, unsubstituted or substituted C₁-C₆ alkyl, or C₁-C₆ haloalkyl. In one embodiment, each of R^{a}, R^{b}, R^{c}, R^{d} and R^{e} is H. In some other embodiments, each of R^{a} and R^{b} is H and at least one of R^{c}, R^{d} and R^{e} is independently halogen (e.g., fluoro, chloro) or unsubstituted C₁-C₆ alkyl (e.g., methyl, ethyl, isopropyl, isobutyl, or t-butyl). For example, R′ is unsubstituted C₁-C₆ alkyl and each of R^{d} and R^{e} is H. In another example, R′ is H and one or both of R^{d} and R^{e} is halogen or unsubstituted C₁-C₆ alkyl. Non-limiting embodiments of the 3′ blocking group include

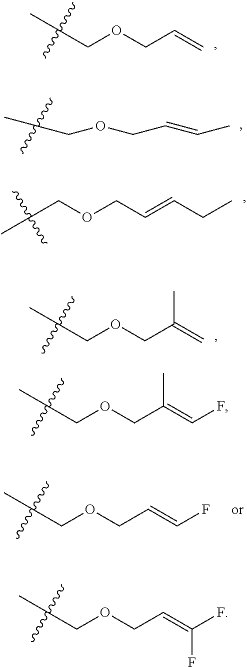

In one embodiment, the 3′ blocking group is

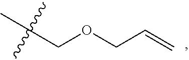 [SMILES: *COCC=C]

and together with the 3′ oxygen it forms

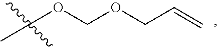 [SMILES: *OCOCC=C]

(“AOM”) group attached to the 3′ carbon atom of the ribose or deoxyribose moiety. Additional embodiments of the 3′ blocking groups are described in U.S. Publication No. 2020/0216891 A1, which is incorporated by reference in its entirety and includes additional examples of 3′ blocking groups such as

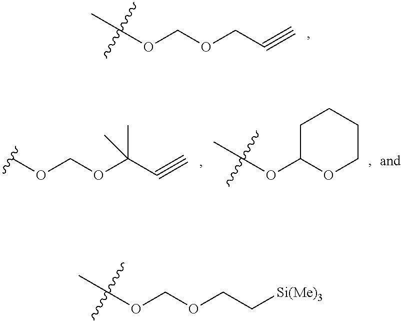

attached to the 3′ carbon atom of the ribose or deoxyribose moiety. In any embodiments of the nucleotide described herein, the nucleotide may comprise a 3′ blocked 2-deoxyribose moiety. Furthermore, the nucleotide may be a nucleoside triphosphate.

Labeled Nucleotides

In some embodiments, the 3′ blocked nucleotide also comprises a detectable label and such nucleotide is called a labeled nucleotide or a fully functionalized nucleotide (ffN). The label (e.g., a fluorescent dye) is conjugated via a cleavable linker by a variety of means including hydrophobic attraction, ionic attraction, and covalent attachment. In some aspect, the dyes are conjugated to the nucleotide by covalent attachment via the cleavable linker. One of ordinary skill in the art understands that label may be covalently bounded to the linker by reacting a functional group of the label (e.g., carboxyl) with a functional group of the linker (e.g., amino). In some such embodiments, the cleavable linker may comprise a moiety that is the same as the 3′ blocking group. As such, the cleavable linker and the 3′ blocking group may be cleaved or removed under the same reaction condition. In some such embodiments, the cleavable linker may comprise an allyl moiety, more particularly comprises a moiety of the structure:

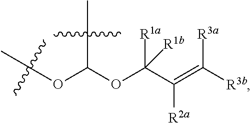

wherein each of R^{1a}, R^{1b}, R^{2a}, R^{3a} and R^{3b} is independently H, halogen, unsubstituted or substituted C₁-C₆ alkyl, or C₁-C₆ haloalkyl.

In some embodiments, the dye may be covalently attached to oligonucleotides or nucleotides via the nucleotide base. For example, the labeled nucleotide or oligonucleotide may have the label attached to the C5 position of a pyrimidine base or the C7 position of a 7-deaza purine base through a cleavable linker moiety.

Nucleotides may be labeled at sites on the sugar or nucleobase. As known in the art, a “nucleotide” consists of a nitrogenous base, a sugar, and one or more phosphate groups. In RNA, the sugar is ribose and in DNA is a deoxyribose, i.e., a sugar lacking a hydroxy group that is present in ribose. The nitrogenous base is a derivative of purine (e.g., deazapurine, 7-deazapurine) or pyrimidine. The purines are adenine (A) and guanine (G), and the pyrimidines are cytosine (C) and thymine (T) or in the context of RNA, uracil (U). The C-1 atom of deoxyribose is bonded to N−1 of a pyrimidine or N−9 of a purine. A nucleotide is also a phosphate ester of a nucleoside, with esterification occurring on the hydroxy group attached to the C-3 or C-5 of the sugar. Nucleotides are usually mono, di- or triphosphates.

Although the base is usually referred to as a purine or pyrimidine, the skilled person will appreciate that derivatives and analogues are available which do not alter the capability of the nucleotide or nucleoside to undergo Watson-Crick base pairing. “Derivative” or “analogue” means a compound or molecule whose core structure is the same as, or closely resembles that of a parent compound but which has a chemical or physical modification, such as, for example, a different or additional side group, which allows the derivative nucleotide or nucleoside to be linked to another molecule. For example, the base may be a deazapurine. In particular embodiments, the derivatives should be capable of undergoing Watson-Crick pairing. “Derivative” and “analogue” also include, for example, a synthetic nucleotide or nucleoside derivative having modified base moieties and/or modified sugar moieties. Such derivatives and analogues are discussed in, for example, Scheit, Nucleotide analogs (John Wiley & Son, 1980) and Uhlman et al., Chemical Reviews 90:543-584, 1990. Nucleotide analogues can also comprise modified phosphodiester linkages including phosphorothioate, phosphorodithioate, alkyl-phosphonate, phosphoranilidate, phosphoramidite linkages and the like.

In particular embodiments the labeled nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit. The spacer distances, for example, the nucleotide base from a cleavage site or label.

The disclosure also encompasses polynucleotides incorporating a nucleotide described herein. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

In some embodiments, the labeled nucleotide described herein comprises or has the structure of Formula (I):

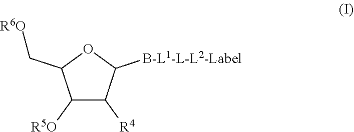

wherein B is the nucleobase;
R⁴ is H or OH;
R⁵ is an allyl containing 3′ blocking group, such as

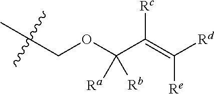

as described herein or a phosphoramidite;
R⁶ is H, monophosphate, diphosphate, triphosphate, thiophosphate, a phosphate ester analog, a reactive phosphorous containing group, or a hydroxy protecting group;
L is an allyl moiety containing linker, such as

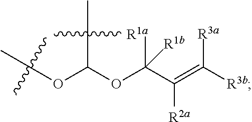

and
each of L¹ and L² is independently an optionally present linker moiety.

In some embodiments of the nucleotide described herein, each of R^{1a}, R^{1b}, R^{2a}, R^{3a} and R^{3b} is H. In other embodiments, at least one of R^{1a}, R^{1b}, R^{2a}, r^{3a} and R^{3b} is halogen (e.g., fluoro, chloro) or unsubstituted C₁-C₆ alkyl (e.g., methyl, ethyl, isopropyl, isobutyl, or t-butyl). In some such instances, each of R^{1a} and R^{1b} is H and at least one of R^{2a}, R^{3a} and R^{3b} is unsubstituted C₁-C₆ alkyl or halogen (for example, R^{2a} is unsubstituted C₁-C₆ alkyl and each of R^{3a} and R^{3b} is H; or R^{2a} is H and one or both of R^{3a} and R^{3b} is halogen or unsubstituted C₁-C₆ alkyl). In one embodiment, the cleavable linker or L comprises

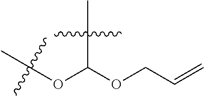 [SMILES: *OC(*)OCC=C]

(“AOL” linker moiety).

In some embodiments of the nucleotide described herein, the nucleobase (“B” in Formula (I)) is purine (adenine or guanine), a deaza purine, or a pyrimidine (e.g., cytosine, thymine or uracil). In some further embodiments, the deaza purine is 7-deaza purine (e.g., 7-deaza adenine or 7-deaza guanine). Non-limiting examples of B comprises

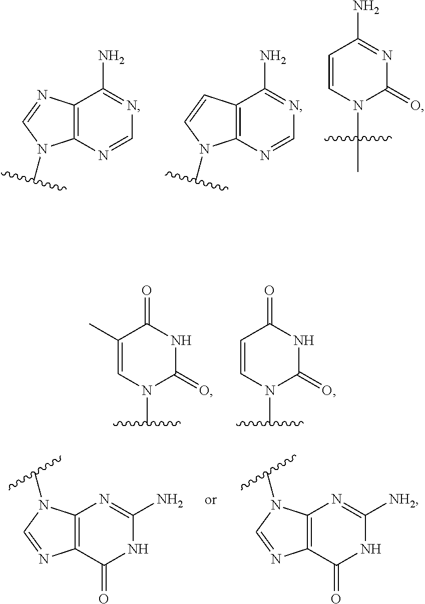

or optionally substituted derivatives and analogs thereof. In some further embodiments, the labeled nucleobase comprises the structure

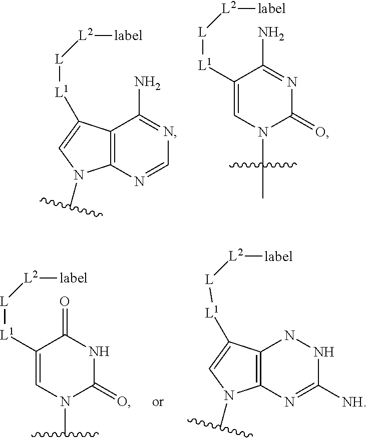

In some other embodiments of the nucleotide described herein, R⁵ in Formula (I) is a phosphoramidite. In such embodiments, R⁶ is an acid-cleavable hydroxy protecting group which allows subsequent monomer coupling under automated synthesis conditions.

In some embodiments of the nucleotide described herein, L¹ is present and L¹ comprises a moiety selected from the group consisting of a propargylamine, a propargylamide, an allylamine, an allylamide, and optionally substituted variants thereof. In some further embodiments, L¹ comprises

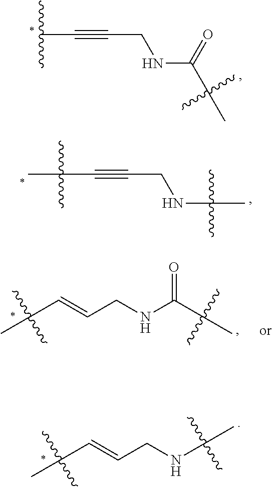 [SMILES: *C#CCNC(*)=O ; *C#CCN* ; */C=C/CNC(*)=O ; */C=C/CN*]

In some further embodiments, the asterisk * indicates the point of attachment of L¹ to the nucleobase (e.g., C5 position of a pyrimidine base or the C7 position of a 7-deaza purine base).

In some embodiments, the nucleotide described herein is a fully functionalized nucleotide (ffN) comprises a 3′-OH blocking group described herein and a dye compound covalently attached to the nucleobase through the cleavable linker described herein, where the cleavable linker comprises L¹ of the structure

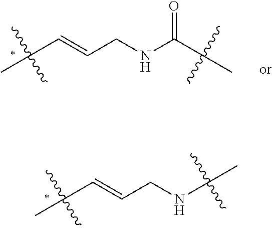 [SMILES: */C=C/CNC(*)=O ; */C=C/CN*]

and * indicates the point of attachment of L¹ to the nucleobase (e.g., C5 position of cytosine, thymine or uracil base, or the C7 position of 7-deaza adenine or 7-deaza guanine). In some instances, ffNs with the allylamine or allylamide linker moiety described herein is also called ffN-DB, ffN-db, ffN-(DB) or ffN-(db), where “DB” or “db” both refer to the double bond in the linker moiety. In some instances, sequencing runs with ffNs set (including ffA, ffT, ffC and ffG) where one or more ffNs is ffN-DB provide superior incorporation rate of the ffNs as compared to the ffNs set with propargylamine or propargylamide linker moiety (also known as ffN-PA or ffN-(PA)) described herein. For example, ffNs-DB set with allylamine or allylamide linker moiety and 3′-AOM blocking group described herein may confer at least 5%, 10%, 20%, 30%, 40%, 50%, 60%, 70%, 80%, 90%, 100%, 200%, 300%, 400%, or 500%, improvement on incorporation rate compared to the ffNs-PA set with 3′-O-azidomethyl blocking group at the same condition for the same period of time, thereby improve phasing values. In other embodiments, the incorporation rate/speed is measured by surface kinetics Vmax on the surface of a substrate (e.g., a flow cell or cBot system). For example, ffNs-DB set with 3′-AOM blocking group may confer at least 5%, 10%, 20%, 30%, 40%, 50%, 60%, 70%, 80%, 90%, 100%, 200%, 300%, 400%, or 500%, improvement on Vmax value (ms⁻¹) compared to the ffNs-PA set with 3′-O-azidomethyl blocking group at the same condition for the same period of time. In some embodiments, the incorporation rate/speed is measured at ambient temperature or a temperature below ambient temperature (such as 4-10° C.). In other embodiments, the incorporation rate/speed is measured at an elevated temperature, such as 40° C., 45° C., 50° C., 55° C., 60° C. or 65° C. In some such embodiments, the incorporation rate/speed is measured in solution in a basic pH environment, e.g., at pH 9.0, 9.2, 9.4, 9.6, 9.8 or 10.0. In some such embodiments, the incorporation rate/speed is measured with the presence of an enzyme, such as a polymerase (e.g., a DNA polymerase), a terminal deoxynucleotidyl transferase, or a reverse transcriptase. In some embodiments, the ffN-DB is ffT-DB, ffC-DB or ffA-DB. In one embodiment, the ffNs-DB set with improved phasing value described herein comprises ffT-DB, ffC, ffA and ffG. In another embodiment, the ffNs-DB set with improved phasing value described herein comprises ffT-DB, ffC-DB, ffA and ffG. In yet another embodiment, the ffNs-DB set with improved phasing value described herein comprises ffT-DB, ffC-DB, ffA-DB and ffG.

In some further embodiments, when the nucleobase of the nucleotide described herein is thymine or optionally substituted derivatives and analogs thereof (i.e., the nucleotide is T), L¹ comprises an allylamine moiety or an allylamide moiety, or optionally substituted variants thereof. In particular examples, L¹ comprises

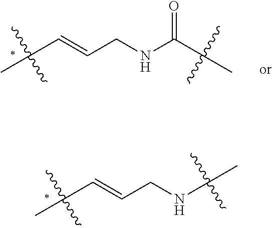 [SMILES: */C=C/CNC(*)=O ; */C=C/CN*]

and * indicates the point of attachment of L¹ to the C5 position of the thymine base. In some embodiments, the T nucleotide described herein is a fully functionalized T nucleotide (ffT) labeled with a dye molecule through the cleavable linker comprising

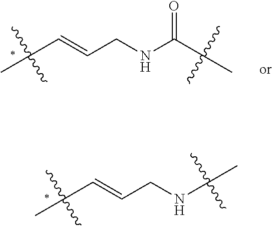 [SMILES: */C=C/CNC(*)=O ; */C=C/CN*]

directly attached to the C5 position of the thymine base (i.e., ffT-DB). In some instances, when ffT-DB is used in sequencing applications in the presence of a palladium catalyst, it may substantially improve sequencing metrics such as phasing, pre-phasing and error rate. For example, when ffT-DB with 3′-AOM blocking group described herein is used, it may confer at least 50%, 100%, 200%, 300%, 400%, 500%, 600%, 700%, 800%, 900%, 1000%, 1500%, 2000%, 2500%, or 3000% improvement on one or more sequencing metrics described herein compared to when a standard ffT-PA with 3′-O-azidomethyl blocking group is used.

Some further embodiments of the nucleoside or nucleotide described herein include those with Formula (Ia), (Ia′), (Ib), (Ic), (Ic′) or (Id):

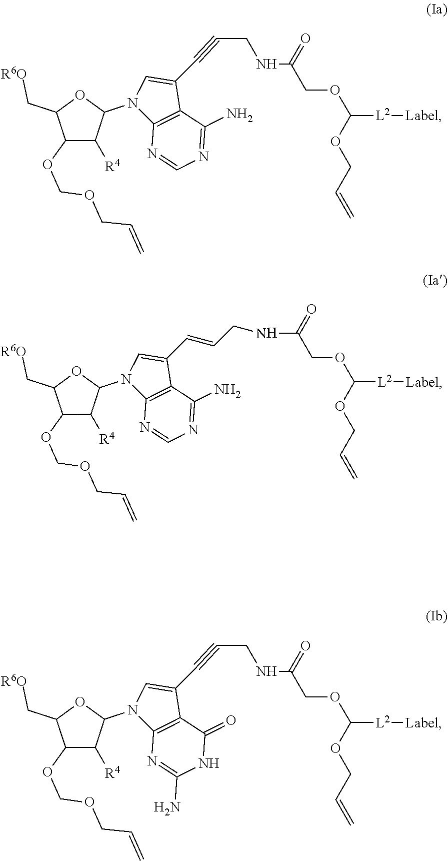

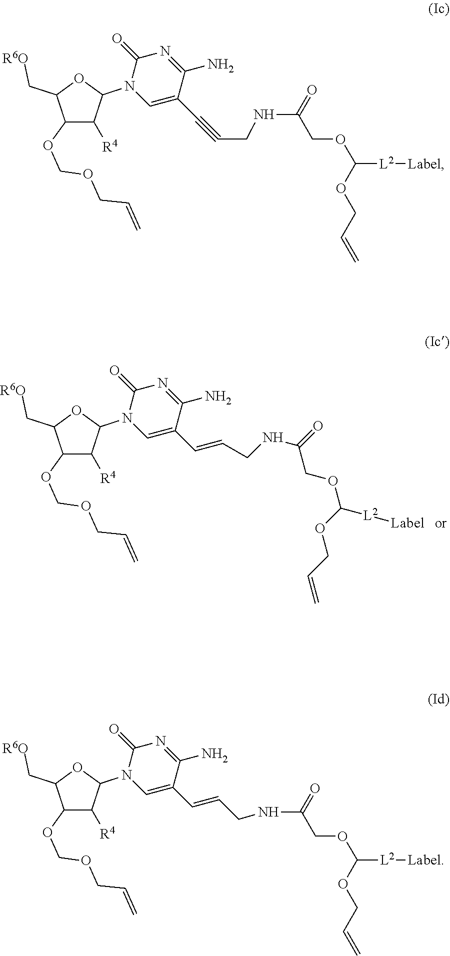

In some further embodiments of the nucleotide described herein, L² is present and L² comprises

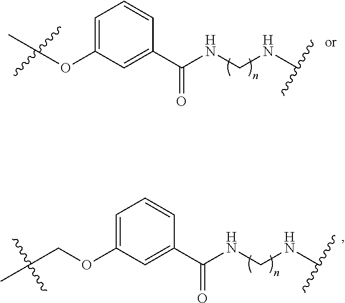

wherein n is an integer of 1, 2, 3, 4, 5, 6, 7, 8, 9 or 10 and the phenyl moiety is optionally substituted. In some such embodiments, n is 5 and the phenyl moiety of L² is unsubstituted.

In any embodiments of the nucleotide described herein, the cleavable linker or L¹/L² may further comprise a disulfide moiety or azido moiety (such as

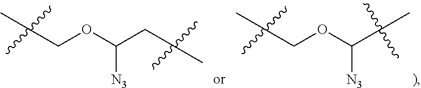 [SMILES: *COC(C*)N=[N+]=[N-] ; *COC(*)N=[N+]=[N-]]

or a combination thereof. Additional non-limiting examples of a linker moiety may be incorporated into L¹ or L² include:

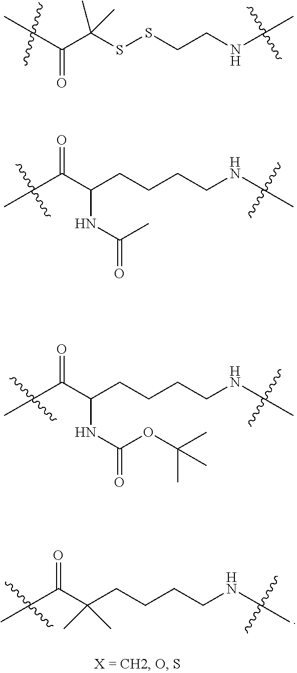 [SMILES: *NCCSSC(C)(C)C(*)=O ; *NCCCCC(NC(C)=O)C(*)=O ; *NCCCCC(NC(=O)OC(C)(C)C)C(*)=O ; *NCCCCC(C)(C)C(*)=O]

Additional linker moieties are disclosed in WO 2004/018493 and U.S. Publication No. 2016/0040225, which are herein incorporated by references.

Non-limiting exemplary labeled nucleotides as described herein include:

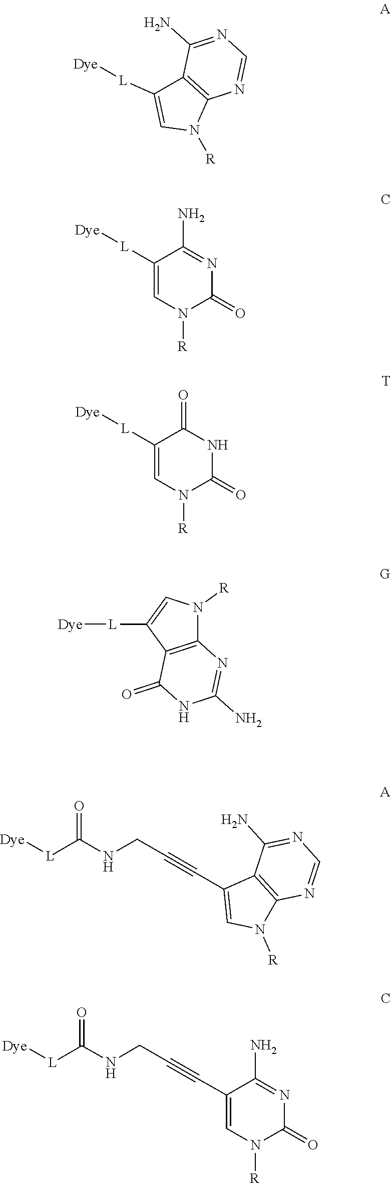

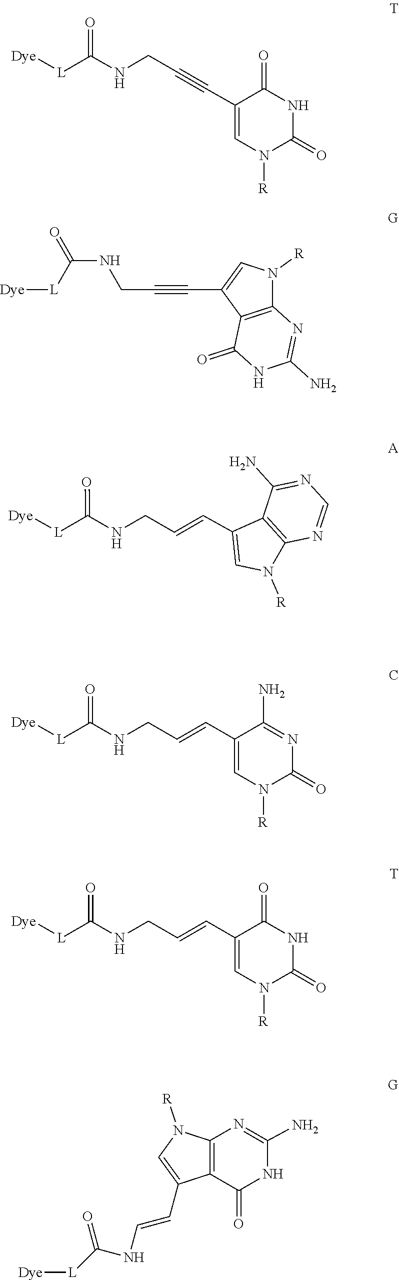

wherein L represents a cleavable linker (optionally include L² described herein) and R represents a ribose or deoxyribose moiety as described above, or a ribose or deoxyribose moiety with the 5′ position substituted with one, two or three phosphates.

In some embodiments, non-limiting exemplary fluorescent dye conjugates are shown below:

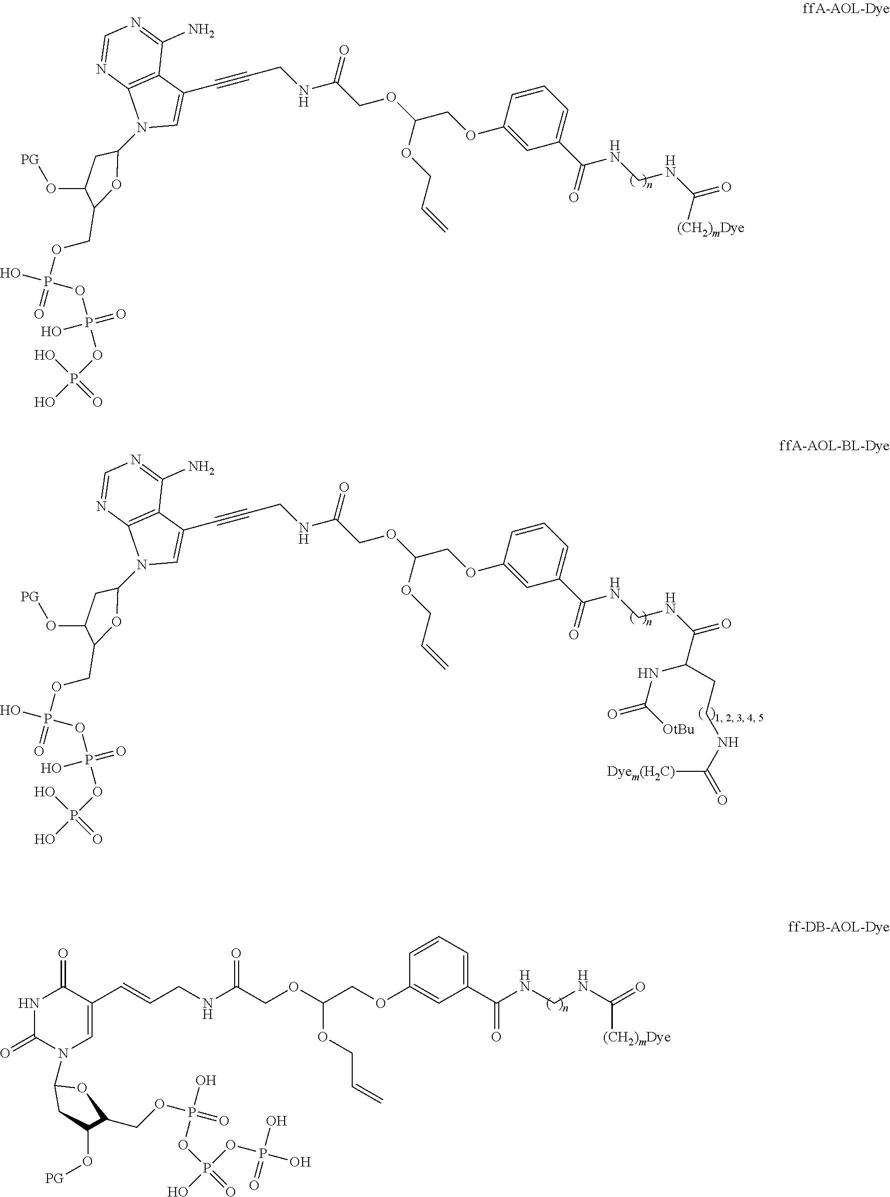

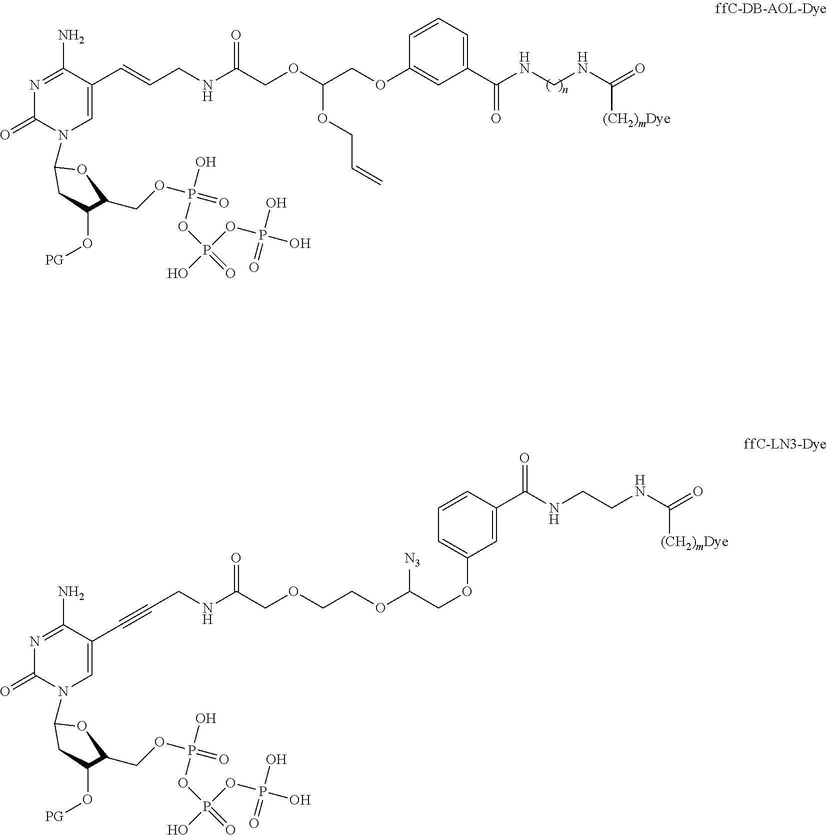

wherein PG stands for the 3′ blocking groups described herein; n is an integer of 1, 2, 3, 4, 5, 6, 7, 8, 9, or 10; and m is 0, 1, 2, 3, 4, or 5. In one embodiment, —O-PG is AOM. In one embodiment, n is 5.

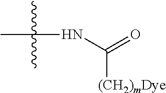

refers to the connection point of the Dye with the cleavable linker as a result of a reaction between an amino group of the linker moiety and the carboxyl group of the Dye.

Various fluorescent dyes may be used in the present disclosure as detectable labels, in particularly those dyes that may be excitation by a blue light (e.g., about 450 nm to about 460 nm) or a green light (e.g., about 520 nm to about 540 nm). These dyes may also be referred to as “blue dyes” and “green dyes” respectively. Examples of various type of blue dyes, including but not limited to coumarin dyes, chromenoquinoline dyes, and bisboron containing heterocycles are disclosed in U.S. Publication Nos. 2018/0094140, 2018/0201981, 2020/0277529, 2020/0277670, 2021/0188832 and 2022/0033900, and U.S. Ser. Nos. 17/550,271, 17/736,688, and 63/325,057, each of which is incorporated by reference in its entirety. Examples of green dyes including cyanine or polymethine dyes disclosed in International Publication Nos. WO2013/041117, WO2014/135221, WO 2016/189287, WO2017/051201 and WO2018/060482A1, each of which is incorporated by reference in its entirety.

In any embodiments of nucleotide described herein, the nucleotide comprises a 2′ deoxyribose moiety (i.e., R⁴ is Formula (I) and (Ia)-(Id)) is H). In some further respect, the 2′ deoxyribose contains one, two or three phosphate groups at the 5′ position of the sugar ring. In some further aspect, the nucleotides described herein are nucleotide triphosphate (i.e., —OR⁶ is Formula (I) and (Ia)-(Id)) forms triphosphate).

Additional embodiments of the present disclosure relate to an oligonucleotide or a polynucleotide comprising a nucleoside or nucleotide described herein. In some such embodiments, the oligonucleotide or polynucleotide is hybridized to a template or target polynucleotide. In some such embodiments, the template polynucleotide is immobilized on a solid support.

Additional embodiments of the present disclosure relate to a solid support comprises an array of a plurality of immobilized template or target polynucleotides and at least a portion of such immobilized template or target polynucleotides is hybridized to an oligonucleotide or a polynucleotide comprising a nucleoside or nucleotide described herein.

The present application will also be further described with reference to DNA, although the description will also be applicable to RNA, PNA, and other nucleic acids, unless otherwise indicated.

Cleavage Condition of the Cleavable Linker

In any embodiments of the nucleotides or nucleosides described herein, the 3′ blocking group and the cleavable linker (and the attached label) may be removable under the same or substantially same chemical reaction conditions, for example, the 3′ blocking group and the detectable label may be removed in a single chemical reaction. In other embodiments, the 3′ blocking group and the detectable labeled are removed in two separate steps.

The cleavable linker described herein may be removed or cleaved under various chemical conditions. Non-limiting cleaving condition includes a palladium catalyst, such as a Pd(II) complex (e.g., Pd(OAc)₂, allylPd(II) chloride dimer [(Allyl)PdCl]₂ or Na₂PdCl₄) in the presence of a phosphine ligand, for example tris(hydroxylpropyl)phosphine or tris(hydroxymethyl)phosphine. In some embodiments, the 3′ blocking group may be cleaved under the same or substantially the same cleavage condition as that for the cleavable linker.

Compatibility with Linearization

In order to maximize the throughput of nucleic acid sequencing reactions it is advantageous to be able to sequence multiple template molecules in parallel. Parallel processing of multiple templates can be achieved with the use of nucleic acid array technology. These arrays typically consist of a high-density matrix of polynucleotides immobilized onto a solid support material.

WO 98/44151 and WO 00/18957 both describe methods of nucleic acid amplification which allow amplification products to be immobilized on a solid support in order to form arrays comprised of clusters or “colonies” formed from a plurality of identical immobilized polynucleotide strands and a plurality of identical immobilized complementary strands. Arrays of this type are referred to herein as “clustered arrays.” The nucleic acid molecules present in DNA colonies on the clustered arrays prepared according to these methods can provide templates for sequencing reactions, for example as described in WO 98/44152. The products of solid-phase amplification reactions such as those described in WO 98/44151 and WO 00/18957 are so-called “bridged” structures formed by annealing of pairs of immobilized polynucleotide strands and immobilized complementary strands, both strands being attached to the solid support at the 5′ end. In order to provide more suitable templates for nucleic acid sequencing, it is preferred to remove substantially all or at least a portion of one of the immobilized strands in the “bridged” structure in order to generate a template which is at least partially single-stranded. The portion of the template which is single-stranded will thus be available for hybridization to a sequencing primer. The process of removing all or a portion of one immobilized strand in a “bridged” double-stranded nucleic acid structure is referred to as “linearization.” There are various ways for linearization, including but not limited to enzymatic cleavage, photo-chemical cleavage, or chemical cleavage. Non-limiting examples of linearization methods are disclosed in PCT Publication No. WO 2007/010251, U.S. Patent Publication No. 2009/0088327, U.S. Patent Publication No. 2009/0118128, and U.S. Publication No. 2019/0352327, which are incorporated by reference in their entireties.

In some embodiments, the condition for the removal of the 3′ blocking group and/or the cleavable linker is also compatible with the linearization processes, for example, a chemical linearization process which comprises the use of a Pd complex and a phosphine. In some embodiments, the Pd complex is a Pd(II) complex (e.g., Pd(OAc)₂, [(Allyl)PdCl]₂ or Na₂PdCl₄), which generates Pd(0) in situ in the presence of the phosphine (e.g., THP).

### Embodiments and Alternatives of Sequencing-By-Synthesis

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina, Inc.), and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

Some embodiments can utilize detection of four different nucleotides using fewer than four different labels. For example, SBS can be performed utilizing methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some other embodiments of sequencing method involve the use the 3′ blocked nucleotide described herein in nanoball sequencing technique, such as those described in U.S. Pat. No. 9,222,132, the disclosure of which is incorporated by reference. Through the process of rolling circle amplification (RCA), a large number of discrete DNA nanoballs may be generated. The nanoball mixture is then distributed onto a patterned slide surface containing features that allow a single nanoball to associate with each location. In DNA nanoball generation, DNA is fragmented and ligated to the first of four adapter sequences. The template is amplified, circularized and cleaved with a type II endonuclease. A second set of adapters is added, followed by amplification, circularization and cleavage. This process is repeated for the remaining two adapters. The final product is a circular template with four adapters, each separated by a template sequence. Library molecules undergo a rolling circle amplification step, generating a large mass of concatemers called DNA nanoballs, which are then deposited on a flow cell. Goodwin et al., “Coming of age: ten years of next-generation sequencing technologies,” Nat Rev Genet. 2016; 17(6):333-51.

Some embodiments can utilize methods involving the real-time monitoring of DNA polymerase activity. Nucleotide incorporations can be detected through fluorescence resonance energy transfer (FRET) interactions between a fluorophore-bearing polymerase and 7-phosphate-labeled nucleotides as described, for example, in U.S. Pat. Nos. 7,329,492 and 7,211,414, both of which are incorporated herein by reference, or nucleotide incorporations can be detected with zero-mode waveguides as described, for example, in U.S. Pat. No. 7,315,019, which is incorporated herein by reference, and using fluorescent nucleotide analogs and engineered polymerases as described, for example, in U.S. Pat. No. 7,405,281 and U.S. Pub. No. 2008/0108082, both of which are incorporated herein by reference. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in U.S. Pub. Nos. 2009/0026082; 2009/0127589; 2010/0137143; and 2010/0282617, all of which are incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in U.S. Pub. No. 2010/0111768 and U.S. patent application Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in U.S. patent application Ser. No. 13/273,666, which is incorporated herein by reference.

Arrays in which polynucleotides have been directly attached to silica-based supports are those for example disclosed in WO 00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO 2005/047301 (incorporated herein by reference). A still further example of solid-supported template polynucleotides is where the template polynucleotides are attached to hydrogel supported upon silica-based or other solid supports, for example, as described in WO 00/31148, WO 01/01143, WO 02/12566, WO 03/014392, U.S. Pat. No. 6,465,178 and WO 00/53812, each of which is incorporated herein by reference.

A particular surface to which template polynucleotides may be immobilized is a polyacrylamide hydrogel. Polyacrylamide hydrogels are described in the references cited above and in WO 2005/065814, which is incorporated herein by reference. Specific hydrogels that may be used include those described in WO 2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Templates that are to be sequenced may form part of an “array” on a solid support, in which case the array may take any convenient form. Thus, the method of the disclosure is applicable to all types of high-density arrays, including single-molecule arrays, clustered arrays, and bead arrays. Labeled nucleotides of the present disclosure may be used for sequencing templates on essentially any type of array, including but not limited to those formed by immobilization of nucleic acid molecules on a solid support.

However, labeled nucleotides of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO 00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using the nucleotides labeled with dye compounds of the disclosure.

The labeled nucleotides of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO 00/06770 and WO 01/57248, each of which is incorporated herein by reference. Although one use of the nucleotides of the disclosure is in sequencing-by-synthesis reactions, the utility of the nucleotides is not limited to such methods. In fact, the nucleotides may be used advantageously in any sequencing methodology which requires detection of fluorescent labels attached to nucleotides incorporated into a polynucleotide.

In particular, the labeled nucleotides of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

Thus, the present disclosure also encompasses labeled nucleotides which are dideoxynucleotides lacking hydroxy groups at both of the 3′ and 2′ positions, such dideoxynucleotides being suitable for use in Sanger type sequencing methods and the like.

Labeled nucleotides of the present disclosure incorporating 3′ blocking groups, it will be recognized, may also be of utility in Sanger methods and related protocols since the same effect achieved by using dideoxy nucleotides may be achieved by using nucleotides having 3′-OH blocking groups: both prevent incorporation of subsequent nucleotides. Where nucleotides according to the present disclosure, and having a 3′ blocking group are to be used in Sanger-type sequencing methods it will be appreciated that the dye compounds or detectable labels attached to the nucleotides need not be connected via cleavable linkers, since in each instance where a labeled nucleotide of the disclosure is incorporated; no nucleotides need to be subsequently incorporated and thus the label need not be removed from the nucleotide.

In any embodiments of the methods described herein, the nucleotide used in the sequencing application is a 3′ blocked nucleotide described herein, for example, the nucleotide of Formula (I) and (Ia)-(Id). In any embodiments, the 3′ blocked nucleotide is a nucleoside triphosphate.

### Kits

The present disclosure also provides kits for use with a sequencing apparatus, comprising: one or more different types of nucleotides (e.g., four different types of nucleotides from A, T, C and G or U; dATP, dTTP, dCTP and dGTP or dUTP), wherein each of the nucleotides comprises a 3′ blocking group comprising an allyl moiety, such as a 3′ blocking group having the structure

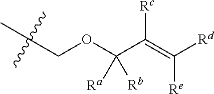

attached to the 3′ oxygen of the nucleotide, wherein each of R^{a}, R^{b}, R^{c}, R^{d} and R^{e} is independently H, halogen, unsubstituted or substituted C₁-C₆ alkyl, or C₁-C₆ haloalkyl; and one or more palladium scavengers, wherein at least one palladium scavenger comprises one or more allyl moieties (e.g., —O-allyl, —S-allyl, —NR-allyl or —N⁺RR′-allyl, or combinations thereof) as described herein.

In some embodiments, the 3′ blocking group in each of the nucleotides in the kit is

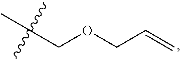 [SMILES: *COCC=C]

and together with the 3′ oxygen forms

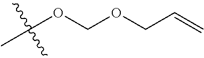 [SMILES: *OCOCC=C]

(“AOM”) group attached to the 3′ carbon atom of ribose or deoxyribose moiety. In some embodiments, the one or more different types of nucleotides are the labeled nucleotides described herein, for example, the 3′ blocked nucleotide of Formula (I), (Ia), (Ia′), (Ib), (Ic), (Ic′) or (Id). In a particular embodiment, a kit can include at least one labeled 3′ blocked nucleotide together with labeled or unlabeled nucleotides. For example, nucleotides labeled with dyes may be supplied in combination with unlabeled or native nucleotides, and/or with fluorescently labeled nucleotides or any combination thereof. Combinations of nucleotides may be provided as separate individual components (e.g., one nucleotide type per vessel or tube) or as nucleotide mixtures (e.g., two or more nucleotides mixed in the same vessel or tube).

In other embodiments, the nucleotides are unlabeled, and the kit may be used with a set of affinity reagents comprising one or more detectable labels, as described herein.

In some embodiments, the Pd scavenger comprising one or more —O-allyl or allyl moieties may be selected from:

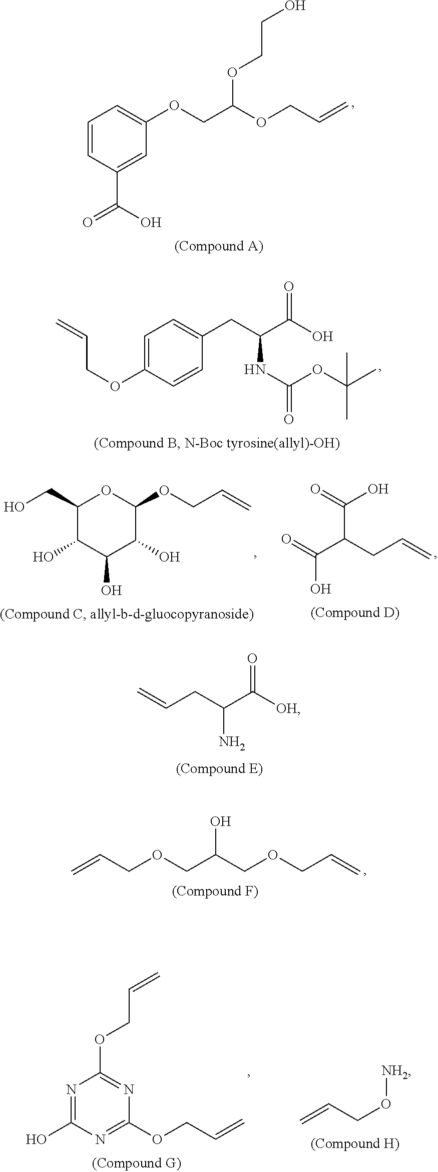

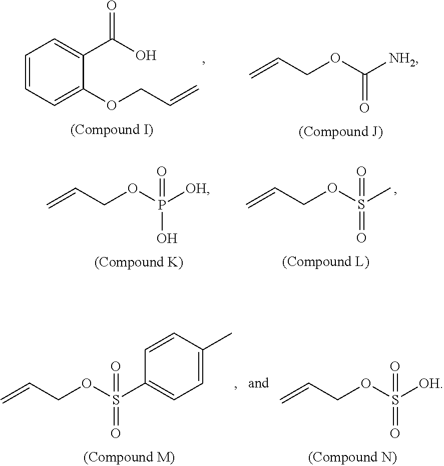

In one embodiment, the kit comprises Compound A. In another embodiment, the kit comprises Compound B. In another embodiment, the kit comprises Compound C.

In some embodiments, the Pd scavenger comprising one or more —S-allyl moieties may be selected from:

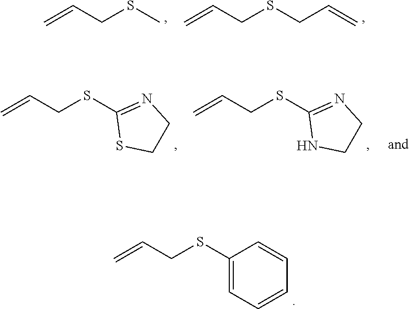

In some embodiments, the palladium scavenger comprising one or more —NR-allyl or —N⁺RR′-allyl moieties may be selected from

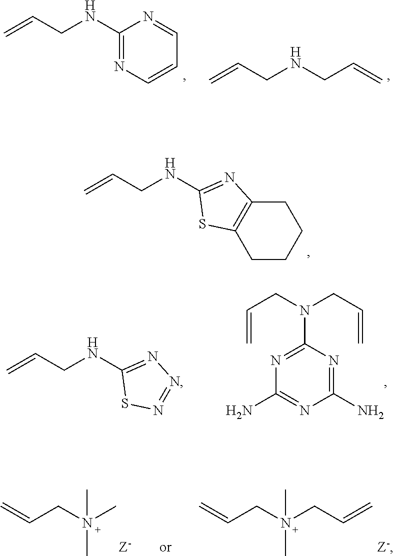

where Z⁻ is an anion (e.g., a halide anion such as F⁻ or Cl⁻). In one embodiment, the kit comprises the palladium scavenger

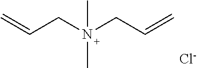

(Compound O, diallyldimethylammonium chloride, also known as DADMAC).

In some embodiments, the kit may be used in the incorporation step of the method described herein. In such embodiment, the kit may comprise additional reagents such as a DNA polymerase such as a mutant of 9° N polymerase, for example, Pol 812, Pol 1901, Pol 1558 or Pol 963. The amino acid sequences of Pol 812, Pol 1901, Pol 1558 or Pol 963 DNA polymerases. In some embodiments, the kit may comprise one or more nucleotides or labeled nucleotides as described herein (A, C, T and G or U; dATP, dCTP, dTTP and dGTP or dUTP). In some embodiments, the kit may also comprise one or more buffering agents. For example, the one or more buffering agents may comprise a primary amine, a secondary amine, a tertiary amine, a natural amino acid, or a non-natural amino acid, or combinations thereof. In further embodiments, the buffering agents comprise ethanolamine or glycine, or a combination thereof. In one embodiment, the buffering agent comprises or is glycine. In some embodiments, the kit may further comprise additional Pd scavenger(s) described herein, such as a Pd(II) scavenger for inactivating a Pd(II) species (e.g., L-cysteine or a salt thereof, or a thiosulfate salt such as sodium thiosulfate). In some embodiments, the Pd(II) scavenger is in a separate compartment from the Pd(0) scavenger. For example, Pd(0) scavenger is in the incorporate mix and the Pd(II) scavenger is in the post cleavage wash solution. In other embodiments, the Pd(0) and Pd(II) scavengers are in the same compartment.

In some embodiments, the components or reagents in the kit are in a dry or lyophilized state, and the kit does not contain any aqueous solution. As such, the reagents in the kit are to be reconstituted to a buffer solution. For example, the DNA polymerase and/or one or more four types of nucleotides may be in a dry or lyophilized form, which are to be reconstituted to form an incorporation mixture (e.g., a first aqueous solution). In some such embodiments, the Pd scavenger comprising one or more allyl moieties as described herein (e.g., Pd(0) scavenger) is also in a dry or lyophilized form, either premixed with the DNA polymerase and/or the nucleotides or in a separate container/compartment and to be reconstituted and mixed with the polymerase and the nucleotides to form the first aqueous solution shortly prior to or at the start of the sequencing runs. In further embodiments, the Pd(II) scavenger may also be in a dry or lyophilized form, premixed with the DNA polymerase and/or the nucleotides. In other embodiments, the Pd(0) scavenger is not in a dry or lyophilized form, and is stored separately from the DNA polymerase and/or the nucleotides and is mixed with an incorporation mixture containing the DNA polymerase and nucleotides to form the first aqueous solution. In other embodiments, the Pd(II) scavenger may be either in the post cleavage wash solution or be in a dry or lyophilized state to be reconstituted in the post cleavage wash solution. In other embodiments, the components in the kit may be provided a concentrated form to be diluted prior to use. In such embodiments a suitable dilution buffer may also be included. In other embodiments, the components of the kit are in a ready to use a buffer solution (e.g., the first aqueous solution or the second aqueous solution). In some embodiments, the first or the second solution has a pH of about 9.

Where kits comprise a plurality, particularly two, or three, or more particularly four, 3′ blocked nucleotides labeled with a detectable label such as a dye compound, the different nucleotides may be labeled with different dye compounds, or one may be dark, with no dye compounds. Where the different type of nucleotides are labeled with different dye compounds, it is a feature of the kits that the dye compounds are spectrally distinguishable fluorescent dyes. As used herein, the term “spectrally distinguishable fluorescent dyes” refers to fluorescent dyes that emit fluorescent energy at wavelengths that can be distinguished by fluorescent detection equipment (for example, a commercial capillary-based DNA sequencing platform) when two or more such dyes are present in one sample. In some embodiments, when two or more nucleotides labeled with fluorescent dye compounds are supplied in kit form, it is a feature of some embodiments that the labeled nucleotides can be excited at the same wavelength, such as, for example by the same laser. In one such feature, three types of nucleotides can be excited by the same wavelength, and the fourth type of nucleotide is unlabeled (dark). In another feature, two types of the labeled nucleotides can be excited at a first wavelength and two types of labeled nucleotides can be excited at a second wavelength. In yet another feature, one type of labeled nucleotides can be excited at a first wavelength, a second type of labeled nucleotides can be excited at a second wavelength, and a third labeled nucleotide can be excited at both the first and the second wavelength. Furthermore, the fourth type of nucleotide is unlabeled. For example, ffC can be excited at the first wavelength, ffT can be excited at a second wavelength, ffA can be excited at both the first and the second wavelengths, and ffG is unlabeled (dark). Particular excitation wavelengths are about 450-460 nm, about 490-500 nm, or about 530-540 nm (e.g., about 532 nm).

In other embodiments, the kits may contain four labeled 3′ blocked nucleotides (e.g., A, C, T, and G or U; dATP, dCTP, dTTP and dGTP or dUTP), where each type of nucleotide comprises the same 3′ blocking group and a fluorescent label, and wherein each fluorescent label has a distinct fluorescence maximum and each of the fluorescent labels is distinguishable from the other three labels. The kits may be such that two or more of the fluorescent labels have a similar absorbance maximum but different Stokes shift. In some other embodiments, one type of the nucleotide is unlabeled.

The present disclosure also provides a cartridge for use with a sequencing apparatus, comprising a plurality of chambers, wherein one of the plurality of the chambers is for use with a kit described herein (e.g., an incorporation mix kit for the incorporation step of the sequencing method described herein). Again, one or more of the components identified in a method set forth herein can be included in a kit. The scan mix, the cleavage mix, or the post-cleavage washing solution described herein may each be in the form of a kit designed to be used in separate chambers of a sequencing cartridge described herein.

### EXAMPLES

Additional embodiments are disclosed in further detail in the following examples, which are not in any way intended to limit the scope of the claims.

### Example 1. Pd Scavenger Compound A Used in Sequencing-by-Synthesis on iSeq™ Platform

In this experiment, the efficiency of a Pd scavenger (Compound A) was tested on Illumina's iSeq™ against a standard SBS run with lipoic acid in the post cleavage wash solution. Sequencing was performed using two-excitation/one-emission SBS (or 2Ex SBS) using on-board iSeq™ Blue (˜450 nm) and green (˜520 nm) excitation sources. PhiX was used as sequencing template. Isothermal 60° C. 2Ex SBS sequencing recipe was used, including standard reuse, 35 s incorporation and 10 s cleavage time using Na₂PdCl₄ cleave mix, RTA data were analyzed using Illumina Sequencing Analysis software. Compound A was added to the incorporation mix (IMX) at various concentrations—0.5 mM, 1 mM, 2 mM, and 10 mM. Addition components of the incorporation mix include: (1) a set of nucleotides comprising ffC-db-AOM-AOL-Dye 1, ffA-db-AOM-AOL-Dye 2, ffT-db-AOM-AOL-NR550S0, and pppG-AOM (dark G); (2) DNA polymerase Poly 1901; and (3) glycine buffer. For the sequencing run using lipoic acid as Pd scavenger, two post-cleavage wash steps were performed. Lipoic acid at 20 mM was in a first post cleavage wash solution, and a second post-cleavage wash solution containing 10 mM L-cysteine was used to wash away the remaining lipoic acid and the inactive Pd(II) prior to the next cycle. For the sequencing run using Compound A as the Pd(0) scavenger, the lipoic acid containing first post-cleavage washing solution was replaced by the L-cysteine containing second post-cleavage wash solution, thereby reducing the number of reagents in the sequencing run. Dye 1 is a coumarin dye disclosed in U.S. Publication No. 2018/0094140, having the structure moiety

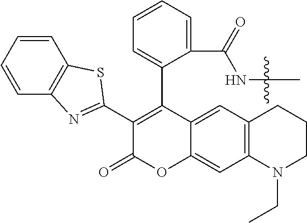 [SMILES: *NC(=O)c1ccccc1-c1c(-c2nc3ccccc3s2)c(=O)oc2cc3c(cc12)CCCN3CC]

when conjugated with the ffC. Dye 2 is a chromenoquinoline dye disclosed in U.S. Ser. No. 17/550,271, which is incorporated by reference, having the structure moiety

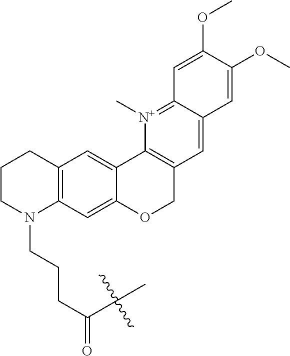 [SMILES: *C(=O)CCCN1CCCc2cc3c(cc21)OCc1cc2cc(OC)c(OC)cc2[n+](C)c1-3]

when conjugated with the ffA. NR550S0 is a known green dye, disclosed in WO2014/135221 A1, which is incorporated by reference.

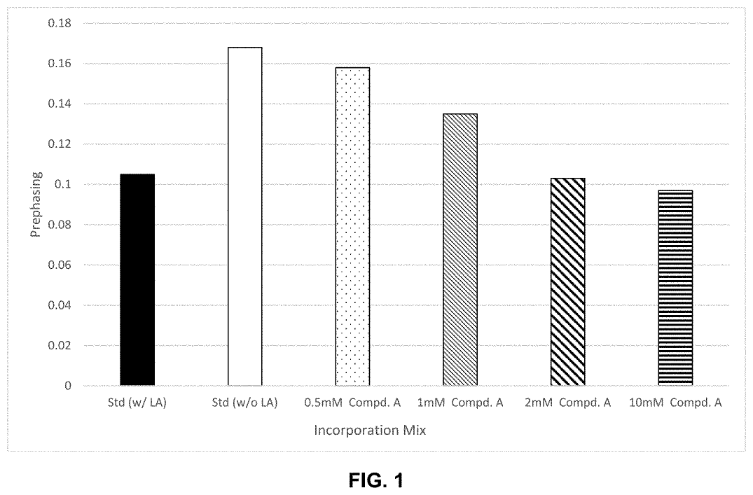

As shown in FIG. 1, when no lipoic acid was used in the post-cleavage wash buffer, the prephasing value was much higher. Adding Compound A to the incorporation mix was able to improve prephasing. When 2 mM or 10 mM Compound A was used in the incorporation mix, the prephasing value was comparable to the standard sequencing run where lipoic acid was used in the first post-cleavage wash step.

### Example 2. Kinetic Evaluation of Various Pd Scavengers

This experiment was conducted for the purpose of evaluating the inhibition capacity of the Pd scavengers when competing with a standard substrate (pA-AOM-NR7180A).

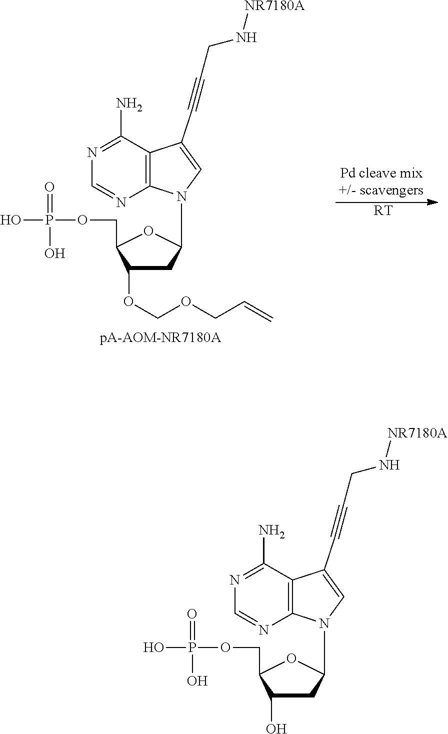

The scavenger candidates were made up into standard buffer solutions (100-25 mM depending on solubility) and adjusted to pH 9.1±0.75. In an Eppendorf tube was added H₂O (148.8 μL), 2 M DEEA (20 μL, pH 9.4), the scavenger stock solution (40-160 μL depending on concentration of stock), a buffer solution containing NaCl, EDTA, Tris and Tween-20 (0-120 μL depending on concentration of scavenger stock), 0.76 mM pA-AOM-NR7180A (51.2 μL). Lastly, a 10 mM [allylPdCl]₂ cleave mix (20 μL) was added to start the reaction. Each reaction mixture had a total volume of 400 μL, and the following composition: 0.1 M DEEA, 10 mM scavenger, 0.1 mM pA-AOM-NR7180A, and 1 mM Pd. At each time point, 40 μL of the solution were immediately quenched with 10 μL of a 1:1 mixture of EDTA/H₂O₂ (0.25:0.25 M) and conversion of pA-AOM-NR7180A in 3′OH-pA-NR7180A were analysed by UPLC. NR7180 is a known rhodamine dye disclosed in U.S. Pat. No. 8,754,244, which is incorporated by reference in its entirety.

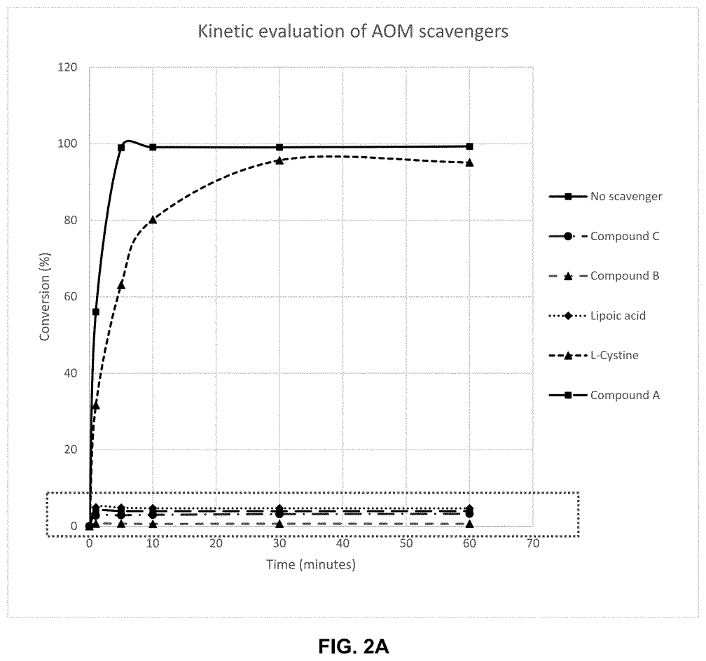

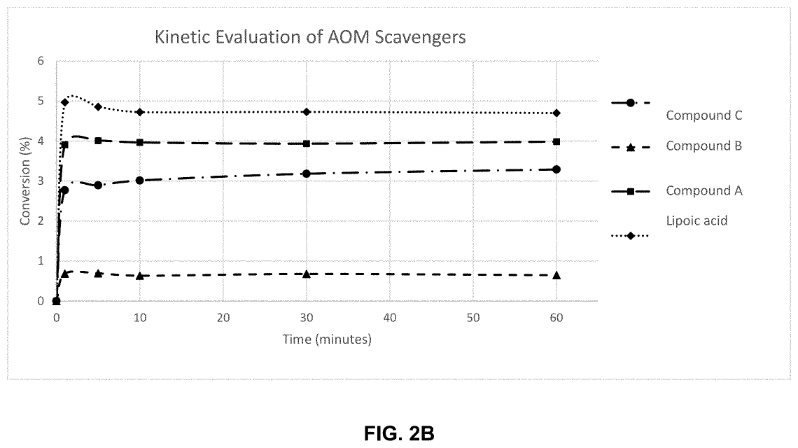

As shown in FIGS. 2A and 2B, Compounds A, B and C showed similar efficient inhibition of the Pd(0) species as compared to lipoic acid, limiting the conversion to 4% and below.

### Example 3. Pd Scavenger Compounds B and C Used in Sequencing-by-Synthesis on iSeq™ Platform

In this experiment, the efficiency of two Pd scavengers (Compound B and Compound C) were tested on Illumina's iSeq™ against a standard SBS run using lipoic acid in the post cleavage wash solution, following similar conditions described above in Example 1. Sequencing recipe was slightly different using 69° C. isothermal, no re-use, 24 s incorporation time and 5.8 s cleavage time. Compound B or C was added to the incorporation mix at 2 mM. Addition components of the incorporation mix include: (1) a set of nucleotides comprising ffC-db-AOM-AOL-Dye 1, ffA-db-AOM-AOL-Dye 2, ffT-db-AOM-AOL-NR550S0, and pppG-AOM (dark G); (2) DNA polymerase Poly 1901; and (3) a glycine buffer. For the sequencing run using lipoic acid, two post-cleavage wash steps were performed. Lipoic acid at 20 mM was in a first post cleavage wash solution, and a second post-cleavage wash solution containing 10 mM L-cysteine was used to wash away the remaining lipoic acid and Pd(II) prior to the next cycle. For the sequencing run using Compound B or C as the Pd(0) scavenger, the lipoic acid containing first post-cleavage washing solution was replaced by the L-cysteine containing second post-cleavage wash solution, thereby reducing the number of reagents in the sequencing run.

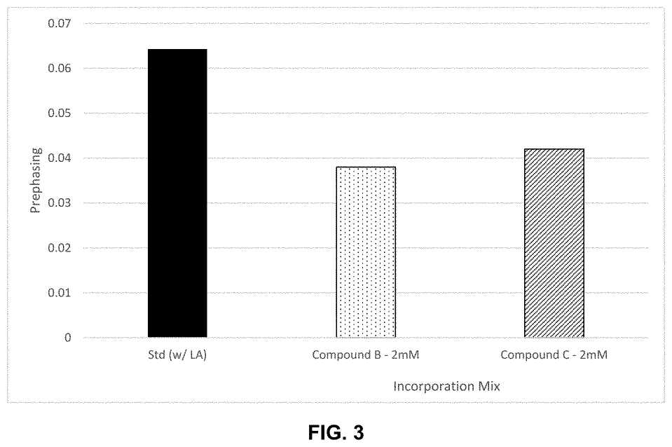

As shown in FIG. 3, adding Compound B or Compound C to the incorporation mix were able to improve the prephasing metrics as compared to using the standard post-cleavage wash steps (first wash solution containing lipoic acid, second wash solution containing L-cysteine).

Additional experiments were conducted by adding 5 mM L-cysteine to the incorporation mix containing Pd scavenger Compound B or C to test the compatibility of L-cysteine in the IMX. In addition, two sequencing recipes were used: 24 s or 19 s incorporation time, 69° C. isothermal, no re-use, and 6 s cleavage time with [allylPdCl]₂ cleave mix. Compound B or C was added to the incorporation mix at 2 mM. Addition components of the incorporation mix include: (1) a set of nucleotides comprising ffC-db-AOM-AOL-Dye 1, ffA-db-AOM-AOL-Dye 2, ffT-db-AOM-AOL-NR550S0, and pppG-AOM (dark G); (2) DNA polymerase Poly 1901; and (3) a glycine buffer. The post-cleavage wash protocol was as follows: (i) first wash solution with 20 mM lipoic acid and second wash solution with 10 mM L-cysteine; (ii) wash solution with 10 mM L-cysteine twice; (iii) and (iv) a standard buffer (an aqueous solution contains Tris, NaCl, EDTA and Tween-20) used in other steps of sequencing (such as clustering and pair end reading) that does not contain L-cysteine.

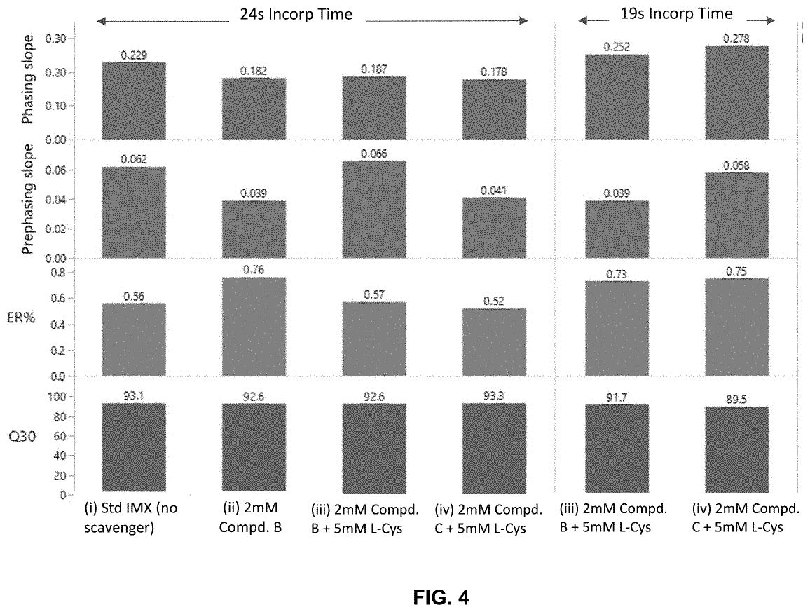

As shown in FIG. 4, it was observed that scavengers in the incorporation mix all gave phasing lower than standard recipe. As expected, a shorter 19 s incorporation time gave a large phasing increase, with increased ER %. Adding L-cysteine to the incorporation mix was able to provide prephasing values that are comparable to that generated from the standard recipe. The main advantage of adding L-cysteine to the incorporation mix is that no separate post-wash reagents/kits need to be developed. A standard buffer solution used in other steps of sequencing (such as clustering and pair end reading) may be used as the post-cleavage wash solution. Furthermore, the sequencing experiments did not show any evidence of negative interaction between the Pd scavengers and other components of the incorporation mix.

In addition to reducing prephasing, inclusion of these scavengers in the incorporation mix also has the potential to suppress the risk of prephasing increase related with cartridge and fluidics cross-contamination.

### Example 4. Pd Scavenger Compound O Used in Sequencing-by-Synthesis on iSeq™ Platform

The efficiency of Pd(0) scavenger diallyldimethylammonium chloride or DADMAC (Compound O) was tested on Illumina's iSeq™ against the previously tested Pd scavenger N-Boc tyrosine (allyl)-OH (Compound B). Sequencing was performed using two-excitation/one-emission SBS (or 2Ex SBS) using on-board iSeq™ Blue (˜450 nm) and green (˜520 nm) excitation sources. PhiX at 100 pM was used as sequencing template. Isothermal 65° C. 2Ex SBS sequencing recipe was used, including standard reuse, 25 s incorporation and 6 s cleavage time using Na₂PdCl₄ cleave mix. RTA data were analyzed using Telescope.

Compound B or Compound O was added to the incorporation mix (IMX) at 2 mM and 0.5 mM respectively. Addition components of the incorporation mix include: (1) a set of nucleotides comprising ffC-db-AOM-AOL-Dye 1, ffA-db-AOM-AOL-Dye 2, ffT-db-AOM-AOL-NR550S0, and pppG-AOM (dark G); (2) DNA polymerase Poly 1901; and (3) glycine buffer. Sodium thiosulfate was added in the post cleavage wash buffer at 10 mM final concentration.

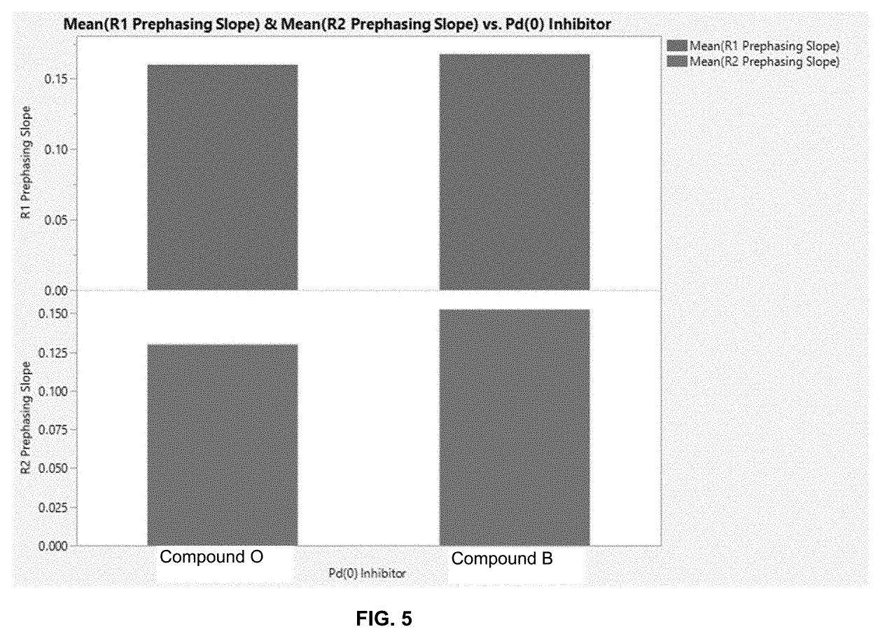

As shown in FIG. 5, adding either Compound O at 0.5 mM or Compound B at 2 mM to the incorporation mix gave comparable prephasing values in the SBS run.

### Example 5. Pd Scavenger Sodium Thiosulfate Used in Sequencing-by-Synthesis on iSeq™ Platform

The efficiency of Pd(II) scavenger sodium thiosulfate was tested on Illumina's iSeq™ against the previously tested Pd(II) scavenger L-cysteine at the same concentration. Sequencing was performed using two-excitation/one-emission SBS (or 2Ex SBS) using on-board iSeq Blue (˜450 nm) and green (˜520 nm) excitation sources. PhiX at 100 pM was used as sequencing template. Isothermal 65° C. 2Ex SBS sequencing recipe was used, including standard reuse, 25 s incorporation and 14 s cleavage time using Na₂PdCl₄ cleave mix. RTA data were analyzed using Telescope.

Pd(0) scavenger Compound B was added to the incorporation mix (IMX) at 2 mM. Addition components of the incorporation mix include: (1) a set of nucleotides comprising ffC-db-AOM-AOL-Dye 1, ffA-db-AOM-AOL-Dye 2, ffT-db-AOM-AOL-NR550S0, and pppG-AOM (dark G); (2) DNA polymerase Poly 1901; and (3) glycine buffer. Either sodium thiosulfate or L-cysteine or nothing was added in the post cleavage wash buffer at 10 mM final concentration.

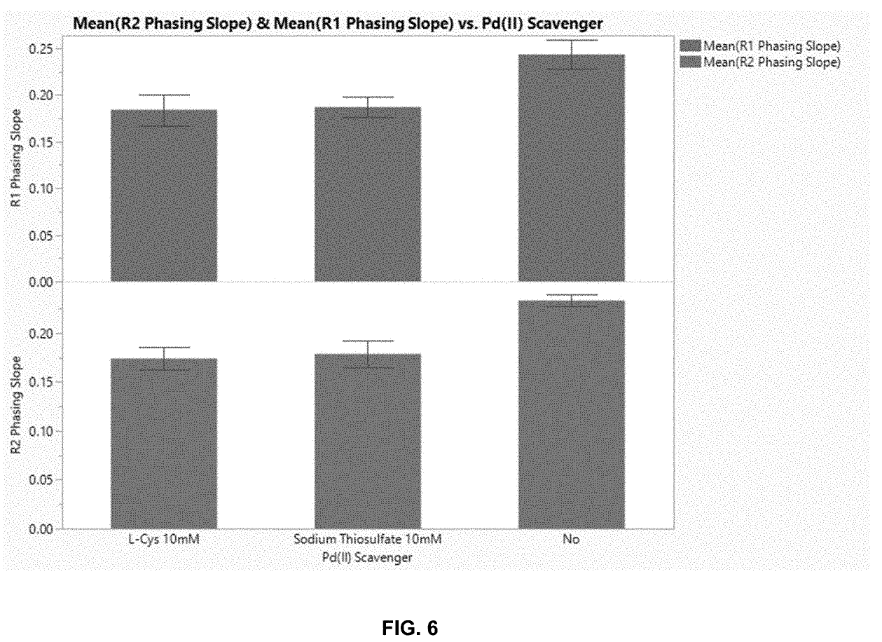

As shown in FIG. 6, when no Pd(II) scavenger was used in post cleavage wash buffer, the phasing value was much higher. Adding either L-cysteine or sodium thiosulfate to the wash buffer was able to improve phasing.

1. A method for determining sequences of a plurality of target polynucleotides, comprising:
(a) contacting a solid support with sequencing primers under hybridization conditions, wherein the solid support comprises a plurality of target polynucleotides immobilized thereon; and the sequencing primers are complementary to at least a portion of the target polynucleotides;
(b) contacting the solid support with a first aqueous solution comprising DNA polymerase and one or more of four different types of nucleotides under conditions suitable for DNA polymerase-mediated primer extension, wherein each of the nucleotides comprises a 3′ blocking group having the structure 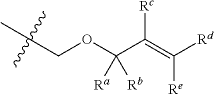
attached to the 3′ oxygen of the nucleotide;
(c) incorporating one type of nucleotides into the sequencing primers to produce extended copy polynucleotides;
(d) performing one or more fluorescent measurements of the extended copy polynucleotides; and
(e) removing the 3′ blocking group of the incorporated nucleotides with a palladium catalyst;
wherein at least a portion of remaining palladium catalyst is inactivated by one or more palladium scavengers, wherein at least one palladium scavenger comprises one or more allyl moieties selected from the group consisting of —O-allyl, —S-allyl, —NR-allyl, and —N⁺RR′-allyl and combinations thereof,
each of R^{a}, R^{b}, R^{c}, R^{d} and R^{e} is independently H, halogen, unsubstituted or substituted C₁-C₆ alkyl, or C₁-C₆ haloalkyl;
R is H, unsubstituted or substituted C₁-C₆ alkyl, unsubstituted or substituted C₂-C₆ alkenyl, unsubstituted or substituted C₂-C₆ alkynyl, unsubstituted or substituted C₆-C₁₀ aryl, unsubstituted or substituted 5 to 10 membered heteroaryl, unsubstituted or substituted C₃-C₁₀ carbocyclyl, or unsubstituted or substituted 5 to 10 membered heterocyclyl; and
R′ is H, unsubstituted C₁-C₆ alkyl or substituted C₁-C₆ alkyl.

2. The method of claim 1, further comprising: repeating steps (b) through (e) until sequences of at least a portion of the target polynucleotides are determined.

3. The method of claim 1, further comprising: (f) washing the solid support with a second aqueous solution after the removal of the 3′ blocking group of the incorporated nucleotides.

4. The method of claim 3, further comprising: repeating steps (b) through (f) until sequences of at least a portion of the target polynucleotides are determined.

5. (canceled)

6. (canceled)

7. The method of claim 1, wherein the palladium scavenger comprising one or more allyl moieties is in the first aqueous solution.

8. The method of claim 7, wherein the concentration of the palladium scavenger comprising one or more allyl moieties in the first aqueous solution is from about 0.1 mM to about 100 mM, from about 0.5 mM to about 50 mM, from about 1 mM to about 20 mM, or from about 2 mM to about 10 mM.

9. The method of claim 3, wherein the palladium scavenger comprising one or more allyl moieties is in the second aqueous solution.

10. The method of claim 9, wherein the concentration of the palladium scavenger comprising one or more allyl moieties in the second aqueous solution is from about 0.1 mM to about 100 mM, from about 0.5 mM to about 50 mM, from about 1 mM to about 20 mM, or from about 2 mM to about 10 mM.

11. The method of claim 1, wherein the palladium scavenger comprising one or more —O-allyl moieties has the structure: 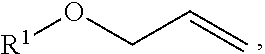
wherein R¹ is C₁-C₁₂ alkyl optionally substituted with one or more R^{x}, C₂-C₁₂ alkenyl optionally substituted with one or more R^{x}, C₂-C₁₂ alkynyl optionally substituted with one or more R^{x}, unsubstituted amino, substituted amino, C₆-C₁₀ aryl, (C₆-C₁₀ aryl) C₁-C₆ alkyl, 5 to 10 membered heteroaryl, (5 to 10 membered heteroaryl) C₁-C₆ alkyl, C₃-C₁₀ carbocyclyl, (C₃-C₁₀ carbocyclyl) C₁-C₆ alkyl, 3 to 10 membered heterocyclyl, (3 to 10 membered heterocyclyl) C₁-C₆ alkyl, a monosaccharide moiety, a disaccharide moiety, an oligosaccharide moiety, an amino acid moiety, —C(═O)NR^{f1}R^{g1}, —P(═O)OR^{f1}OR^{g1}, —C(═O)R^{h1}, —C(═O)OR^{h1} or —S(═O)₂R^{j1}, wherein each of C₆-C₁₀ aryl, 5 to 10 membered heteroaryl, C₃-C₁₀ carbocyclyl and 3 to 10 membered heterocyclyl is optionally substituted with one or more R^{x};
each of R^{f1} and R^{g1} is independently H, C₁-C₆ alkyl optionally substituted with one or more R^{x}, C₆-C₁₀ aryl optionally substituted with one or more R^{x}, or 5 to 10 membered heteroaryl optionally substituted with one or more R^{x};
each R^{h1} is independently C₁-C₆ alkyl optionally substituted with one or more R^{x}, C₆-C₁₀ aryl optionally substituted with one or more R^{x}, or 5 to 10 membered heteroaryl optionally substituted with one or more R^{x};
each R^{j1} is independently hydroxy, C₁-C₆ alkyl optionally substituted with one or more R^{x}, C₆-C₁₀ aryl optionally substituted with one or more R^{x}, or 5 to 10 membered heteroaryl optionally substituted with one or more R^{x}; and
each R^{x} is independently amino, halo, hydroxy, carboxy, cyano, (C₁-C₆ alkyl)amino, C-amido, N-amido, unsubstituted and substituted C₁-C₆ alkyl, C₁-C₆ haloalkyl, unsubstituted and substituted C₁-C₆ alkoxy, C₁-C₆ haloalkoxy, unsubstituted and substituted C₆-C₁₀ aryloxy, sulfo, sulfonate, or —O—CH₂—CH═CH₂.

12. The method of claim 11, wherein the palladium scavenger comprising one or more —O-allyl moieties is selected from the group consisting of: 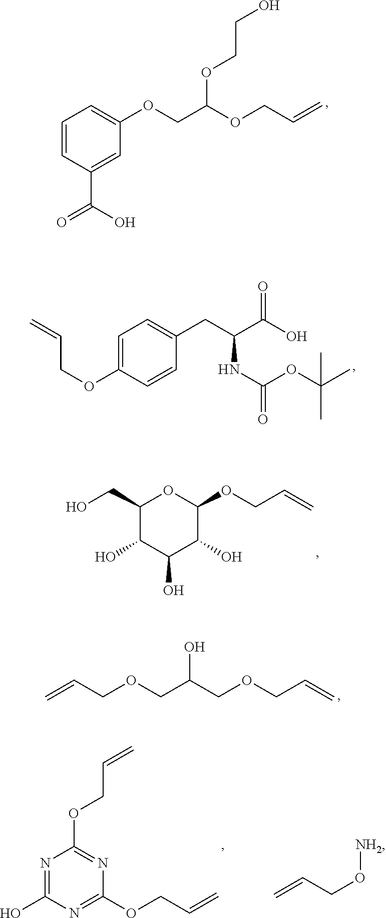 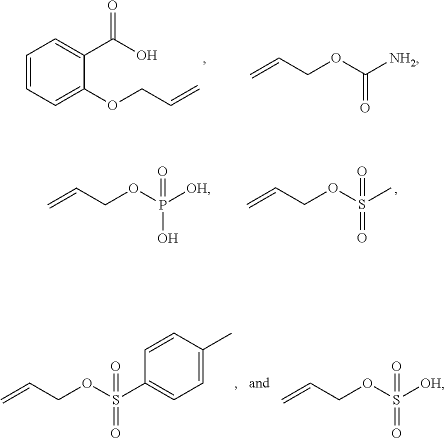
and salts thereof.

13. The method of claim 12, wherein the palladium scavenger comprises 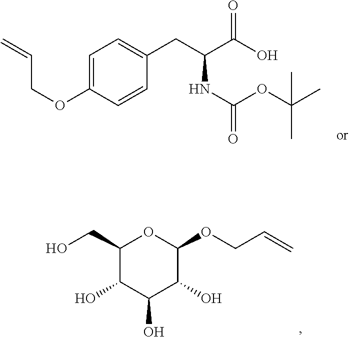 [SMILES: C=CCOc1ccc(C[C@H](NC(=O)OC(C)(C)C)C(=O)O)cc1 ; C=CCO[C@@H]1O[C@H](CO)[C@@H](O)[C@H](O)[C@H]1O]
or a salt thereof.

14. The method of claim 1, wherein the palladium scavenger comprising one or more —S-allyl moieties has the structure: 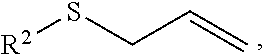
wherein R² is C₁-C₁₂ alkyl optionally substituted with one or more R^{y}, C₂-C₁₂ alkenyl optionally substituted with one or more R^{y}, C₂-C₁₂ alkynyl optionally substituted with one or more R^{y}, unsubstituted amino, substituted amino, C₆-C₁₀ aryl, (C₆-C₁₀ aryl) C₁-C₆ alkyl, 5 to 10 membered heteroaryl, (5 to 10 membered heteroaryl) C₁-C₆ alkyl, C₃-C₁₀ carbocyclyl, (C₃-C₁₀ carbocyclyl) C₁-C₆ alkyl, 3 to 10 membered heterocyclyl, (3 to 10 membered heterocyclyl) C₁-C₆ alkyl, a monosaccharide moiety, a disaccharide moiety, an oligosaccharide moiety, an amino acid moiety, —C(═O)NR^{P}R^{g2}, —P(═O)OR^{f2}OR^{g2}, —C(═O)R^{h2}, —C(═O)OR^{h2} or —S(═O)₂R^{j2}, wherein each of C₆-C₁₀ aryl, 5 to 10 membered heteroaryl, C₃-C₁₀ carbocyclyl and 3 to 10 membered heterocyclyl is optionally substituted with one or more R^{y};
each of R^{f2} and R^{g2} is independently H, C₁-C₆ alkyl optionally substituted with one or more R^{y}, C₆-C₁₀ aryl optionally substituted with one or more R^{y}, or 5 to 10 membered heteroaryl optionally substituted with one or more R^{y};
each R^{h2} is independently C₁-C₆ alkyl optionally substituted with one or more R^{y}, C₆-C₁₀ aryl optionally substituted with one or more R^{y}, or 5 to 10 membered heteroaryl optionally substituted with one or more R^{y};
each R^{j2} is independently hydroxy, C₁-C₆ alkyl optionally substituted with one or more R^{y}, C₆-C₁₀ aryl optionally substituted with one or more R^{y}, or 5 to 10 membered heteroaryl optionally substituted with one or more R^{y}; and
each R^{y} is independently amino, halo, hydroxy, carboxy, cyano, (C₁-C₆ alkyl)amino, C-amido, N-amido, unsubstituted and substituted C₁-C₆ alkyl, C₁-C₆ haloalkyl, unsubstituted and substituted C₁-C₆ alkoxy, C₁-C₆ haloalkoxy, unsubstituted and substituted C₆-C₁₀ aryloxy, sulfo, sulfonate, or —S—CH₂—CH═CH₂.

15. The method of claim 14, wherein the palladium scavenger comprising one or more —S-allyl moieties is selected from the group consisting of:

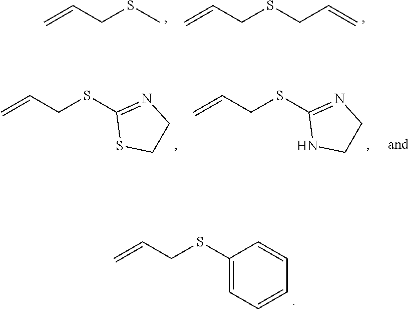

16. The method of claim 1, wherein the palladium scavenger comprising one or more —NR-allyl or —N⁺RR′-allyl moieties having the structure: 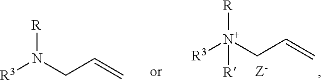
wherein Z is an anion;
each R³ is independently C₁-C₁₂ alkyl optionally substituted with one or more R^{z}, C₂-C₁₂ alkenyl optionally substituted with one or more R^{z}, C₂-C₁₂ alkynyl optionally substituted with one or more R^{z}, unsubstituted amino, substituted amino, C₆-C₁₀ aryl, (C₆-C₁₀ aryl) C₁-C₆ alkyl, 5 to 10 membered heteroaryl, (5 to 10 membered heteroaryl) C₁-C₆ alkyl, C₃-C₁₀ carbocyclyl, (C₃-C₁₀ carbocyclyl) C₁-C₆ alkyl, 3 to 10 membered heterocyclyl, (3 to 10 membered heterocyclyl) C₁-C₆ alkyl, a monosaccharide moiety, a disaccharide moiety, an oligosaccharide moiety, an amino acid moiety, —C(═O)NR^{f3}R^{g3}, —P(═O)OR^{f3}OR^{g3}, —C(═O)R^{h3}, —C(═O)OR^{h3} or —S(═O)₂R^{j3}, wherein each of C₆-C₁₀ aryl, 5 to 10 membered heteroaryl, C₃-C₁₀ carbocyclyl and 3 to 10 membered heterocyclyl is optionally substituted with one or more R^{z};
each of R^{f3} and R^{g3} is independently H, C₁-C₆ alkyl optionally substituted with one or more R^{z}, C₆-C₁₀ aryl optionally substituted with one or more R^{z}, or 5 to 10 membered heteroaryl optionally substituted with one or more R^{z};
each R^{h3} is independently C₁-C₆ alkyl optionally substituted with one or more R^{z}, C₆-C₁₀ aryl optionally substituted with one or more R^{z}, or 5 to 10 membered heteroaryl optionally substituted with one or more R^{z};
each R^{j3} is independently hydroxy, C₁-C₆ alkyl optionally substituted with one or more R^{z}, C₆-C₁₀ aryl optionally substituted with one or more R^{z}, or 5 to 10 membered heteroaryl optionally substituted with one or more R^{z}; and
each R^{z} is independently amino, halo, hydroxy, carboxy, cyano, (C₁-C₆ alkyl)amino, C-amido, N-amido, unsubstituted and substituted C₁-C₆ alkyl, C₁-C₆ haloalkyl, unsubstituted and substituted C₁-C₆ alkoxy, C₁-C₆ haloalkoxy, unsubstituted and substituted C₆-C₁₀ aryloxy, sulfo, sulfonate, or —NH—CH₂—CH═CH₂.

17. The method of claim 16, wherein the palladium scavenger comprising one or more —NR-allyl or —N⁺RR′-allyl moieties is selected from the group consisting of: 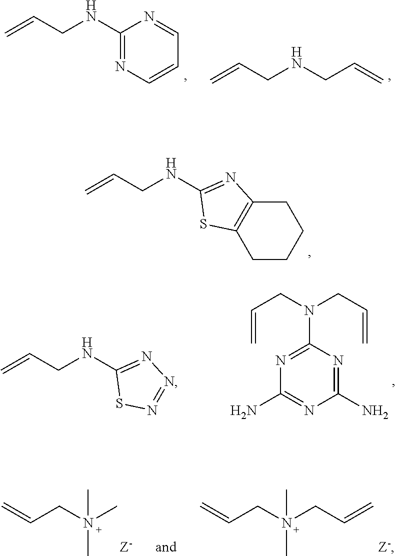
where Z⁻ is Cl⁻ or F⁻.

18. The method of claim 17, wherein the palladium scavenger comprises

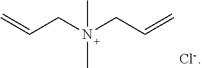

19. The method of claim 1, wherein the 3′ blocking group having the structure 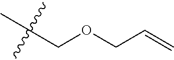 [SMILES: *COCC=C]
attached to the 3′ oxygen of the nucleotide.

20. The method of claim 1, wherein the palladium catalyst is a Pd(0) catalyst generated in situ from a palladium complex and a water-soluble phosphine.

21. The method of claim 20, wherein the palladium complex comprises [Pd(Allyl)Cl]₂, Na₂PdCl₄, K₂PdCl₄, [Pd(Allyl)(THP)]Cl, [Pd(Allyl)(THP)₂]Cl, Pd(CH₃CN)₂C₁₂, Pd(OAc)₂, Pd(PPh₃)₄, Pd(dba)₂, Pd(Acac)₂, PdCl₂(COD), or Pd(TFA)₂, or combinations thereof.

22. (canceled)

23. The method of claim 20, wherein the water-soluble phosphine comprises tris(hydroxypropyl)phosphine (THP), tris(hydroxymethyl)phosphine (THMP), 1,3,5-triaza-7-phosphaadamantane (PTA), bis(p-sulfonatophenyl)phenylphosphine dihydrate potassium salt, tris(carboxyethyl)phosphine (TCEP), or triphenylphosphine-3,3′,3″-trisulfonic acid trisodium salt, or combinations thereof.

24. (canceled)

25. The method of a claim 1, wherein the one or more palladium scavengers further comprises at least one Pd(II) scavenger.

26. The method of claim 25, wherein the Pd(II) scavenger comprises an isocyanoacetate (ICNA) salt, ethyl isocyanoacetate, methyl isocyanoacetate, cysteine or a salt thereof, L-cysteine or a salt thereof, N-acetyl-L-cysteine, a thiosulfate salt, sodium thiosulfate, potassium thiosulfate, potassium ethylxanthogenate, potassium isopropyl xanthate, glutathione, ethylenediaminetetraacetic acid (EDTA), iminodiacetic acid, nitrilodiacetic acid, trimercapto-S-triazine, dimethyldithiocarbamate, dithiothreitol, mercaptoethanol, allyl alcohol, propargyl alcohol, thiol, tertiary amine and/or tertiary phosphine, or combinations thereof.

27. The method of claim 25, wherein the Pd(II) scavenger comprises L-cysteine or sodium thiosulfate.

28. The method of claim 25, wherein the Pd(II) scavenger is in the first aqueous solution or the second aqueous solution, or both.

29. The method of claim 28, wherein the concentration of the Pd(II) scavenger in the first or the second aqueous solution is from about 0.1 mM to about 100 mM, from 0.2 mM to about 75 mM, from about 0.5 mM to about 50 mM, from about 1 mM to about 20 mM, or from about 2 mM to about 10 mM.

30. (canceled)

31. A kit for use with a sequencing apparatus, comprising:
one or more of four different types of nucleotides, wherein each of the nucleotides comprises a 3′ blocking group having the structure 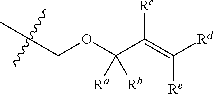
attached to the 3′ oxygen of the nucleotide, wherein each of R^{a}, R^{b}, R^{c}, R^{d} and R^{e} is independently H, halogen, unsubstituted or substituted C₁-C₆ alkyl, or C₁-C₆ haloalkyl; and
one or more palladium scavengers, wherein at least one palladium scavenger comprises one or more allyl moieties selected from the group consisting of —O-allyl, —S— allyl, —NR-allyl and —N⁺RR′-allyl, and combinations thereof,
wherein R is H, unsubstituted or substituted C₁-C₆ alkyl, unsubstituted or substituted C₂-C₆ alkenyl, unsubstituted or substituted C₂-C₆ alkynyl, unsubstituted or substituted C₆-C₁₀ aryl, or unsubstituted or substituted 5 to 10 membered heteroaryl, unsubstituted or substituted C₃-C₁₀ carbocyclyl, or unsubstituted or substituted 5 to 10 membered heterocyclyl; and
R′ is H, unsubstituted C₁-C₆ alkyl or substituted C₁-C₆ alkyl.

32.-39. (canceled)

40. A cartridge for use with a sequencing apparatus, comprising a plurality of chambers, wherein one of the plurality of the chambers is for use with a kit according to claim 31.
